(19) United States
(12) Patent Application Publication
(10) Pub. No.: US 2016/0160231 A1
(43) Pub. Date: Jun. 9, 2016

(54) USE OF POLYPEPTIDES AND NUCLEIC ACIDS FOR IMPROVING PLANT GROWTH, STRESS TOLERANCE AND PRODUCTIVITY

(71) Applicant: Academia Sinica, Taipei (TW)

(72) Inventors: SU-MAY YU, Taipei (TW); Tuan-Hua David Ho, Taipei (TW); Shuen-Fang Lo, Taichung County (TW); Ching-Yi Liao, Taipei (TW)

(73) Assignee: Academia Sinica, Taipei (TW)

(21) Appl. No.: 14/960,915

(22) Filed: Dec. 7, 2015

Related U.S. Application Data
(60) Provisional application No. 62/088,852, filed on Dec. 8, 2014.

(51) Int. Cl. C12N 15/82; C07K 14/415

(52) U.S. Cl. CPC C12N 15/8273; C12N 15/8261; C12N 15/8271; C07K 14/415

(57) ABSTRACT

Transgenic plants that over-express a Big Grain 2 (BG2) gene and methods of using such for improving the growth, productivity, or stress tolerance of a plant.

### RELATED APPLICATION

This application claims the benefit of U.S. provisional application No. 62/088,852, filed Dec. 8, 2014 under 35 U.S.C. §119, the entire content of which is herein incorporated by reference.

### BACKGROUND OF THE INVENTION

The world's crop productivity has reached a plateau in recent years. Breeding of high-yielding rice is crucial for meeting the food demand of the increasing world's population. Plant genetic engineering offers an efficient alternative for increase in crop productivity. Thousands of quantitative trait loci (QTL) have been reported to control yield-related traits. Biological functions of some genes have been validated and used as targets in crop breeding. However, few genes have been found to confer multi functions including plant growth, abiotic stress tolerance and productivity.

### SUMMARY OF THE INVENTION

The present disclosure is based at least in part on the unexpected discovery that overexpression of BIG GRAIN 2 (BG2) gene, including deletion mutants, in a plant such as rice confers enhanced shoot and root growth, increased grain size, longer panicle length, and elevated tolerance to abiotic stress (e.g., osmotic stress, salt, dehydration, or heat) as compared to the wild-type counterpart.

Accordingly, one aspect of the present disclosure features a method for enhancing survival rate of a plant in an environment comprising an abiotic stress factor, the method comprising: (i) providing a transgenic plant (e.g., a monocotyledonous plant or a dicotyledonous plant), which overly expresses a Big Grain 2 (BG2) gene as relative to its wild-type counterpart; and (ii) growing the transgenic plant in an environment comprising an abiotic stress factor, e.g., osmotic stress, salt, dehydration, or heat. Such a transgenic plant is also within the scope of the present disclosure.

Further, the present disclosure provides a method of improving plant growth, architecture, organ size, productivity, or stress tolerance of a plant by over-expressing a Big Grain 2 (BG2) gene in a plant (e.g., those described herein) as relative to its wild-type counterpart.

In some embodiments, the transgenic plant described herein is a monocotyledonous plant, including, but not limited to, Zea mays, Sorghum bicolor, Setaria italica, Hordeum vulgare, Brachypodium distachyon, Oryza sativa, Triticum spp., or Saccharum spp.

In other embodiments, the transgenic plant described herein is a dicotyledonous plant, which includes, but is not limited to, Cucumis sativus, Ricinus communis, Solanum lycopersicum, Solanum tuberosum, Vitis vinifera, Populus trichocarpa, Arabidopsis thaliana, Arabidopsis lyrata, or Platycodon grandiflorus.

In one example, the transgenic plant is a crop, e.g., rice, maize, wheat, barley, sugarcane, banana, cotton, soybean, pea, potato, tomato, brassica, orchid, balloon flower, yam, sweet potato, cassava, rose, petunia, chrysanthemum, lily, or carnation. In another example, the transgenic plant is an angiosperm.

The transgenic plant for use in any of the methods described herein may comprise an exogenous promoter located upstream to an endogenous BG2 gene. An endogenous BG2 gene refers to a native BG2 gene of the plant. Alternatively, the transgenic plant may comprise an exogenous BG2 gene, which is in operable linkage to a suitable promoter, e.g., those described herein.

Any promoter known in the art that is suitable for driving gene expression in plant cells can be used in the transgenic plants and methods described herein. In some embodiments, the promoter may be tissue-specific, growth stage-specific, environment-inducible, stress-inducible, nutrient-inducible or chemical-inducible. In specific embodiments, the promoter is a ubiquitin gene promoter (Ubi) such as a maize ubiquitin gene promoter, an actin promoter such as a rice actin promoter, a CaMV35S promoter, a glutelin promoter such as a rice glutelin promoter, a sporamin promoter such as a sweet potato sporamin promoter, an E8 promoter such as a tomato E8 promoter, a rbcS1 promoter, or an α-amylase promoter.

Also within the scope of the present disclosure is a transgenic plant, which expresses a Big Grain 2 (BG2) protein that comprises an amino acid sequence at least 85% identical to SEQ ID NO:3 or SEQ ID NO:4, wherein the transgenic plant either comprises an exogenous promoter in operable linkage to the endogenous BG2 gene or comprises an exogenous nucleic acid encoding the BG2 protein.

Another aspect of the present disclosure provides an isolated nucleic acid encoding a Big Grain 2 (BG2) mutant, wherein the BG2 mutant contains one or more mutations within one or more of the regions corresponding to Domain 1, Domain 2, Domain 3, Domain 4, and/or Domain 6 in a wild-type BG2 protein (e.g., those set forth as SEQ ID NO:3 or SEQ ID NO:4). In some embodiments, the one or more mutations are deletion(s). For example, the whole region corresponding to Domain 1, Domain 2, Domain 3, Domain 4, or Domain 6 can be deleted in the BG2 mutant.

Also provided herein are (i) a vector (e.g., an expression vector) comprising the nucleic acid described herein that encodes the BG2 mutant; and (ii) a host cell comprising the vector, wherein the host cell can be a plant cell, which can be a monocotyledonous plant cell or a dicotyledonous plant cell; and (iii) a transgenic plant, which expresses the BG2 mutant as described herein. The transgenic plant can be a monocotyledonous plant, which can be Zea mays, Sorghum bicolor, Setaria italica, Hordeum vulgare, Brachypodium distachyon, Oryza sativa, Triticum spp., or Saccharum spp. Alternatively, the transgenic plant can be a dicotyledonous plant, which can be Cucumis sativus, Ricinus communis, Solanum lycopersicum, Solanum tuberosum, Vitis vinifera, Populus trichocarpa, Arabidopsis thaliana, Arabidopsis lyrata, or Platycodon grandiflorus. In some examples, the transgenic plant can be a crop, for example, rice, maize, wheat, barley, sugarcane, banana, cotton, soybean, pea, potato, tomato, brassica, orchid, balloon flower, yam, sweet potato, cassava, rose, petunia, chrysanthemum, lily, or carnation. In other examples, the transgenic plant can be an angiosperm.

In some embodiments, the nucleic acid encoding the BG2 mutant can be in operable linkage to a promoter, which can be a tissue-specific promoter, a growth stage-specific promoter, an environment-inducible promoter, a stress-inducible promoter, a nutrient-inducible prompter, or a chemical-inducible promoter. In some examples, the nucleic acid encoding the BG2 mutant can be in operable linkage to a promoter, which can be a ubiquitin gene promoter (Ubi) such as a maize ubiquitin gene promoter, an actin promoter such as a rice actin promoter, a CaMV35S promoter, a glutelin promoter such as a rice glutelin promoter, a sporamin promoter such as a sweet potato sporamin promoter, an E8 promoter such as a tomato E8 promoter, a rbcS1 promoter, or an α-amylase promoter.

Further, the present disclosure provides a method of improving plant growth, architecture, organ size, productivity, or stress tolerance, the method comprising expressing a Big Grain 2 (BG2) mutant as described herein in a plant.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1. Identification of the rice BG2 gene by the T-DNA activation tagging. The coding sequence of BG2 is 933 bp with no intron. The T-DNA insertion site of D16/BG2act is ˜5.7 kb upstream of BG2.

FIG. 2. Characterization of BG2 amino acid sequence and multiple alignments with its homologs. A: Multiple alignments of BG2-like proteins in seven organisms. Similar and identical amino acid residues are shaded in black and gray background, respectively. B: schematic representation of constructs carrying BG2 truncated at different conserved domains and expressed under the control of the Ubi promoter. WT: wild type rice; FL, full-length BG2; AD, conserved domain-deleted BG2. C: Comparison grain size of the transgenic plants in TNG67 (T2 generation) and Kitaake (T1 generation) background with wide-type. The sequences shown in FIG. 2, panel A, from top to bottom, correspond to SEQ ID NOs:3 and 5-11.

FIG. 3. BG2 encodes a novel protein which is conserved in Angiosperms. A neighbor-joining tree of BG2 and its homologs. The tree was constructed using MEGA 6.0 and bootstrapped with 1000 replicates. The proteins are named according to their gene/EST names or NCBI accession numbers. The length of the branches is proportional to the amino acid variation rates. At, Arabidopsis thaliana; Al, Arabidopsis lyrata; Bd, Brachypodium distachyon; Cs, Cucumis sativus; Hv, Hordeum vulgare; Pt, Populus trichocarpa; Rc, Ricinus communis; Sb, Sorghum bicolor; Si, Setaria italica; Sl, Solanum lycopersicum; St, Solanum tuberosum; Vv, Vitis vinifera; Zm, Zea mays.

FIG. 4. Overexpression of BG2 controlled by the maize ubiquitin promoter leads to phenotypes similar to D16/BG2act lines. Panicle length increase significantly in 2 independent BG2OX lines. (A) Expression level of BG2 in seedlings at 14 d after imbibition (DAI) as detected by RT-PCR. (B) Panicle of wild-type, 2 independent BG2OX lines, and D16/BG2act line. (C) Length of main panicle of wild-type, 2 independent BG2OX lines, and D16/BG2act line. A Student's t-test was used to generate the P values. Asterisks show that significantly different from WT. *, P<0.05; ***, P<0.001.

FIG. 5. Overexpression of BG2 controlled by the maize ubiquitin promoter leads to phenotypes similar to D16/BG2act lines. Grain length, width, and thickness increase significantly in 2 independent BG2OX lines. (A) spikelet before pollination; (B) grain with husk; (C) grain without husk. (D) Grain length, width, and thickness were measured by Vernier caliper (n=30). A Student's t-test was used to generate the P values. Asterisks indicate significantly different from WT. ***, P<0.001. Bar=1 cm.

FIG. 6. The grain yield of transgenic rice cultivar TK2 overexpressing BG2 has greater yield than the wild type. The BG2 cDNA is expressed in transgenic rice cultivar TK2 under the control of the Ubi promoter.

FIG. 7. BG2L may be functionally redundant to BG2 in grain size control. (A) Phenotype of BG2OX, BG2act, and BG2 RNAi line compared to the wild-type. (B) Expression level of BG2 in wild-type and BG2 RNAi seedlings. (C) T-DNA activation insertion mutant of BG2L also shows larger grain. Seeds were screened by hygromycin containing MS medium.

FIG. 8. Cell number but not cell size accounts for the increase in grain length in BG2OX and BG2act lines. Scanning electron microscopic analysis of the outer epidermis of wild-type, BG2OX-1, BG2OX-2, and BG2act, at the central position of the lemma (n=3). The distance between tubercles in the outer epidermis was measured (n=250). No significant difference of cell density was observed between the wild type and BG2 overexpression or activation lines. Total cell number increased in both BG2 overexpression and activation lines. Values are shown with mean±standard deviation.

FIG. 9. BG2 is expressed ubiquitously in various rice tissues and BG2 promoter is active ubiquitously during rice development. (A) Spatial and temporal expression of RBG2 in various organs as determined by RT-PCR. Total RNA was extracted from various rice tissues: leaf bladse of 14-d-old seedlings, shoots and roots of 11-d-old seedlings, basal region of 7-d-old shoot containing shoot apex, 3 cm young panicle, 12 cm young panicle, 21 cm young panicle, flag leaf of 20 cm panicle, flag leaf sheath of 20 cm panicle, rachis branch of 20 cm panicle, spikelet of 20 cm panicle, 2 days after pollination (DAP), 4 DAP, 6 DAP, 8 DAP. (B-N) Histochemical GUS staining of transgenic rice containing pRBG2:eGFP-GUS. (B) Cross section of leaf sheath near node. (C) Root tip of 9 DAI seedling. (D) Initiating lateral root of 9 DAI seedling. (E) Calli induced from seed. (F) Seed section after imbibition for 24 h. (G) Stem of 15 DAI seedling. (H-J) Developing seed after 6-day pollination, 14-day pollination, and 20-day pollination. (K) Second leaf laminar joint of 7 DAI seedling. (L) Internode at reproductive stage. (M-N) 5 mm- and 20 cm-long panicle before heading stage.

FIG. 10. Survival rate of BG2 overexpression transgenic plants under PEG, drought, and salt treatment is higher than the wild-type.

FIG. 11. Transgenic Arabidopsis overexpressing BG2 displays larger organ size. The rice BG2 was fused downstream of the CaMV35S promoter, generating the CaMV35S:BG2 construct. The construct was then introduced into the Arabidopsis genome, and transgenic Arabidopsis was used for following experiments: (A) Accumulation of BG2 mRNA in seedlings at 14 d after imbibition (DAI) as detected by RT-PCR. (B) (i) 5 DAI seedling (ii) 21 DAI seedling (iii) 30 DAI plant (iv) flower (v) seed. (C) Silique length (n=10) (D) seed number per silique (n=10) (E) seed length (n=30) (F) seed width (n=30). ***, P<0.001.

FIG. 12. Transgenic Arabidopsis overexpressing BG2 is salt tolerant. Transgenic Arabidopsis carrying CaMV35S:BG2 (OX1) and wild type (Col) were germinated and grown in medium containing 0, 50, 100, and 150 mM NaCl.

FIG. 13 include charts showing that BG2 confers abiotic stress tolerance in transgenic rice. A: Survival ratio of seedlings after recovery from PEG treatment. B: Survival ratio of seedlings after recovery from NaCl treatment. C: Survival ratio of seedlings after recovery from dehydration. D: Survival ratio of seedlings after recovery from heat treatment. Significance levels with the t-test: * P<0.05, ** P<0.01, *** P<0.001.

### DETAILED DESCRIPTION OF THE INVENTION

Described herein are transgenic plants and methods for improving the growth properties, productivity or stress tolerance of plants by over-expressing a Big Grain 2 (BG2) gene.

As described below in greater detail, over-expressing a BG2 gene increases panicle length, grain size and grain weight in rice, which are considered to be essential traits controlling grain yield. Additionally, over-expressing a BG2 gene confers tolerance to various abiotic stresses (e.g., osmotic stress, salt, dehydration, or heat), which allows the transgenic plants, e.g., those described herein, to cope with global climate/environmental changes.

### I. Big Grain 2 Gene

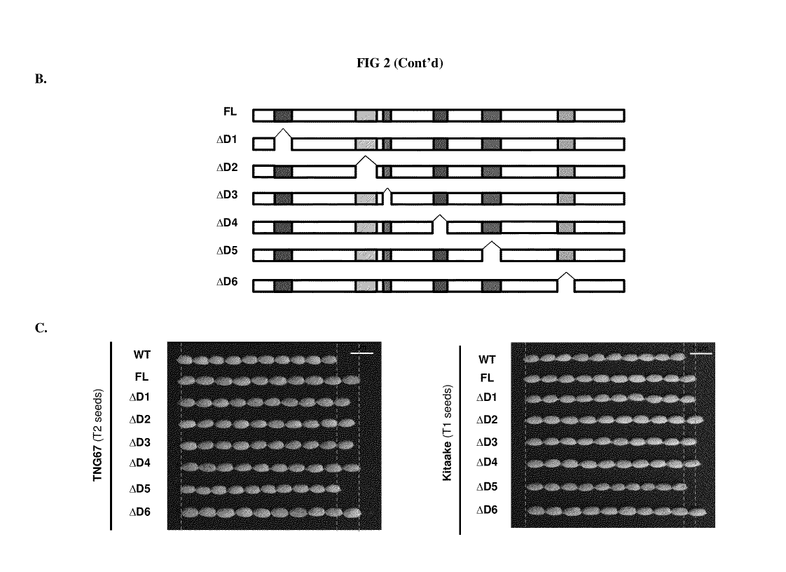

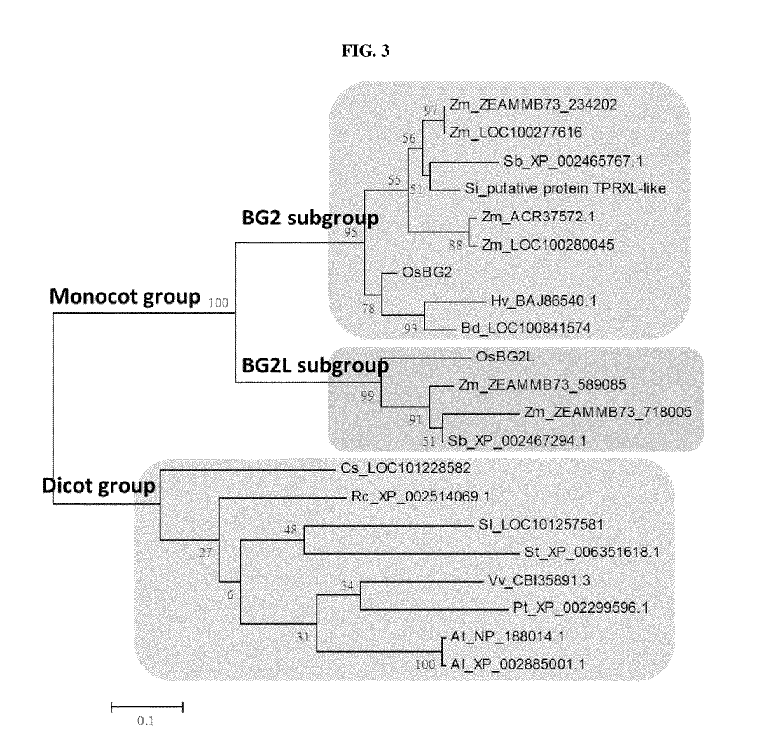

The Big Grain 2 (BG2) gene for use in constructing the transgenic plant described here can be a wild-type BG2 or BG2L genes of Oryza sativa ssp. Japonica cv. Nipponbare described herein, for example, the BG2 protein set forth as SEQ ID NO: 3, the BG2L protein set forth as SEQ ID NO: 4, as well as their orthologs found in other plant species (e.g., those shown in FIG. 2 and FIG. 3).

The full-length cDNA of the BG2 gene from Oryza sativa ssp. Japonica cv. Nipponbare, reported by the Rice Genome Annotation Project database (Os03g0175800), comprises the nucleotide coding sequence of (SEQ ID NO: 1) and encodes a polypeptide of 310 amino acids (SEQ ID NO: 3). The rice genome also encodes a BG2-related protein (BG2L), which has been reported by the Rice Genome Annotation project database (Os10g25810). The BG2L gene comprises the nucleotide coding sequence of (SEQ ID NO: 2), which encodes a polypeptide of 337 amino acids as set forth in (SEQ ID NO: 4). The nucleotide sequences and amino acid sequences of the rice BG2 and BG2L genes/polypeptides are shown below:

```
(SEQ ID NO: 1)
ATGGAGAGGTGGGCGGCGCCCAAGGTGACGGCTGGTTCGGCGAGGCGGTACGTCGCCGACCAGCCGTCTTTCTCATC
CACGCTGCTCGACGCGATATACAAGTCGATGGACGAGCAGCCGGGTCACGGCGGCGGCGCCACCGGGGTGGAGGCGG
TGGCTGCGGCGGCCAAGAAGCAGCACGAGGCGGCCCTGCACTATGGGAACTACTACAAGCCGTCGCTCGCGGGGAGC
TACCGGGCGCGCGCGCCGGGTCCGCACGCCACGACGTCGAGCTCGTCGGAGTGTTCTAGCTACGGCGGGTTCTCGTC
GTCCGAGGCGGAGTCGTCGCACCACCGACGCCTCCGCCCCATCCGCACGACTGTCCCCGGTGGTGCGCCGGGGCCCG
CGCCGGAGAAGAAGGCCAAGAAGCCCGGGGCCTCCATACGCGCCAAGCTCAGGGACCTCCGCAAGCCGGCTTCCCCC
GGCGCGCGCCTCGCGGGCTTCCTCAACTCCATCTTCGCCGGCAAGCGCGCGCCGGCCACGCCGCCCTCGGCGACGGC
CGGGGCGGAGTCCGCGTGCTCGACGGCGTCGTCCTACTCCCGCTCCTGCCTGAGCAAGACGCCGTCGACGCGCGGGC
AGGCGAAGCGGACCGTGCGGTTCTTGGACAGCGACACGGAGTCCCTGGCGTCGTCGACGGTGGTCGACCGCCGCAGG
GTGCCCGTGGAGGCGGTGCAGCAGATGCTGCTCCAGCGGATGGAGATGGAGAGCGACGAGGACGACGACGAGAGCAG
CGACGCGAGCTCCGACCTGTTCGAGCTCGAGAACTTCGCCGCCATTGCTCCCGCCGGGGCCGCGTACCGGGACGAGC
TGCCGGTGTACGAGACGACCAGAGTGGCGCTGAACCGCGCCATTGGCCATGGGTATGGCCATGGACGGAGCGCCAGA
GTTGTCTGA
(SEQ ID NO: 2)
ATGCGCGACATGGAGATGAGGTGGGCGGCGCCGGCGCCGGCGGCGAGGGGGAGGGGGAGGGCGAGGCGGCGGGCGCC
TGACCAGCCGTCGTTCTCGTCGACGCTGCTCGACGCCATCTGCGACTCCATGGACGAGGGCGGCGAGGACGGCCGGA
CAAGAAACGCGGCGAGTGCGGCGGCCAAGAAGAGGCAGGAGGCGGCGAACAGCTACCACTACTACTACTGCTACAAG
CCGTCGCTGGCCGCCAGCTACAGGGCGGCGCCGGCGCTGGGTTCCACGGCGGATTGCCCCGGGAGGGGCTACTTCTC
GTCGTCCGAGGTGGAGTACTCGCTTCGCCGGCTCCGCCCCATCCGCACCTCCGCCGCCGGTGGCGCGGGAGACGGCG
CGGCGGTCGCGCGGAAGCAGCGGCATGAGCAGCCGGATGTGGAGAAGACGGCGAAGACGAAGCCGGGCTCCGCCTCG
GCCCGCGCGTGCCGCAGGCCGGCGTCGCCCGGCGCGCGGCTCGCCAGCTTGCTCAACTCCATCTTCTCCGGCAAGCG
TCCATCCGCGCAGCGCCCGGCGTGTTCGCCGGACTACCCGGAGCCCGCGTGCTCGACGGCGCCACCGTCGTCGTCGT
CGTCGTACGCGCGCCGCCCCTGCCACGCCAAGACGCCGCGCACCCCCCCCACCACCACCACCACGGCGAGGGCGCGG
CCGAGCCGGAGCAGGACCGTCCGGTTCCTGGACATCGACGGCAAGGTCGCGGTGGCCGCGGCCGTCGCCGGCTGCCG
CCGAATTCCGGTCATGGAGGTGGAGGCCGACACCGACGACGGAGGTGAGGAGAGCAGCGACGCGAGCTCGGACCTGT
TCGAGCTCGACAGCCTCGCGGCCATTGCTCCGGCGGGTGGTCGCGACGGCTCCTACGGGGACGAGCTGCCGGTGTAC
GGGACCACCGGAGTTGGGATCCGCCGCGACATTGGCCGCCGCCGTCCGTACGGCCATGCTCCTTGTCGGAGCTGGAG
TAGGGCTGTCTAG
(SEQ ID NO: 3)
MERWAAPKVTAGSARRYVADQPSFSSTLLDAIYKSMDEQPGHGGGATGVEAVAAAAKKQHEAALHYGNYYKPSLAGS
                                                Domain 1
YRARAPGPHATTSSSSECSSYGGFSSSEAESSHHRRLRPIRTTVPGGAPGPAPEKKAKKPGASIRAKLRDLRKPASP
                                    Domain 2 Domain 3
GARLAGFLNSIFAGKRAPATPPSATAGAESACSTASSYSRSCLSKTPSTRGQAKRTVRFLDSDTESLASSTVVDRRR
  Domain 4 Domain 5
VPVEAVQQMLLQRMEMESDEDDDESSDASSDLFELENFAAIAPAGAAYRDELPVYETTRVALNRAIGHGYGHGRSAR
                                                        Domain 6
VV
(SEQ ID NO: 4)
MRDMEMRWAAPAPAARGRGRARRRAPDQPSFSSTLLDAICDSMDEGGEDGRTRNAASAAAKKRQEAANSYHYYYCYK
PSLAASYRAAPALGSTADCPGRGYFSSSEVEYSLRRLRPIRTSAAGGAGDGAAVARKQRHEQPDVEKTAKTKPGSAS
ARACRRPASPGARLASLLNSIFSGKRPSAQRPACSPDYPEPACSTAPPSSSSSYARRPCHAKTPRTPPTTTTTARAR
PSRSRTVRFLDIDGKVAVAAAVAGCRRIPVMEVEADTDDGGEESSDASSDLFELDSLAAIAPAGGRDGSYGDELPVY
GTTGVGIRRDIGRRRPYGHAPCRSWSRAV
```

The BG2 proteins set forth as SEQ ID NO:3 and SEQ ID NO:4 share high sequence homology to Big Grain 1 (Os1_10227) and Big Grain 1 Like proteins (GenBank Accession No. A2Z6Z0 as of Nov. 11, 2015), respectively.

Further, orthologs of BG2 and BG2L are in other cereals, including barley, maize, and sorghum and similar proteins are conserved among several dicotyledonous plants such as Arabidopsis, populous, and Vitis. Examples of such genes found in various plant species are shown in FIG. 3. Other orthologs of BG2 and BG2L proteins can be obtained from available gene databases (e.g., GenBank) using, e.g., SEQ ID NO:3 or SEQ ID NO:4 as a query.

In other embodiments, the BG2 described herein can be a functional variant of a wild-type counterpart, e.g., a functional variant of SEQ ID NO:3 or SEQ ID NO:4. A BG2 functional variant gene can be nucleic acid that encodes a protein having a sufficient level of homology to a wild-type BG2 protein (e.g., the rice BG2 or BG2L gene described herein) and preserves the same biological functionality as the wild-type counterpart. Homology, as used herein, refers to the overall relatedness between nucleic acids (e.g., DNA molecules and/or RNA molecules) or polypeptides. In some embodiments, nucleic acids or proteins are considered to be “homologous” to one another if their sequences are at least 50%, at least 55%, at least 60%, at least 65%, at least 70%, at least 75%, at least 80%, at least 85%, at least 90%, at least 95%, or at least 99% identical. In some examples, a BG2 functional variant may encode a polypeptide sharing at least 75% (e.g., 80%, 85%, 90%, or 95%) sequence identity to the polypeptide encoded by the wild-type counterpart.

Calculation of the percent identity of two amino acid sequences, for example, can be performed by aligning the two sequences for optimal comparison purposes (e.g., gaps can be introduced in one or both of a first and second amino acid sequence for optimal alignment and non-identical sequences can be disregarded for comparison purposes). The amino acids at corresponding amino acid positions are then compared. When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference.

In other embodiments, nucleic acids or proteins are considered to be “homologous” to one another if their sequences are at least 50%, at least 55%, at least 60%, at least 65%, at least 70%, at least 75%, at least 80%, at least 85%, at least 90%, at least 95%, or at least 99% similar. Similarity between two amino acid sequences is defined as the presence of a series of identical as well as conserved amino acid residues in both sequences. The higher the degree of similarity between two amino acid sequences, the higher the correspondence, sameness or equivalence of the two sequences. Suitable programs for calculating the percent identity or similarity between sequences are generally known in the art. As one example, MatGAT (Matrix Global Alignment Tool) is a similarity/identity matrix generator that calculates the similarity and identity between every pair of sequences in a given data set without requiring pre-alignment of the data. The program performs a series of pairwise alignments using the Myers and Miller global alignment algorithm (Myers E W, Miller W. Optimal alignments in linear space. Comp Applic Biosci. 1988; 4:11-17.), calculates similarity and identity, and then places the results in a distance matrix. In order to increase alignment speed, they are computed in the C++ language while the “front-end” of the MatGAT program is encoded in Java.

As shown in FIG. 2, there are six highly conserved regions of the BG2 polypeptides that are shared among Oryza sativa, Arabidopsis thaliana, Arabidopsis lyrata, Brachypodium distachyon, Cucumis sativus, Hordeum vulgare, Sorghum bicolor and Zea mays. These conserved regions are indicated by boxes shown in FIG. 2 and highlighted (boldfaced and underlined) in SEQ ID NO:3. These conserved regions in BG2 proteins of other species can be identified by comparing the sequences of such BG2 proteins with a reference sequence such as SEQ ID NO:3. Studies described herein showed that Domain 5 might be essential to the activity of BG2, e.g., promoting plant growth and/or increasing grain size. Introducing amino acid variations outside of these conserved regions, particularly outside Domain 5, would be expected to have no or less impact on the functionality of the BG2 gene.

In some examples, the BG2 functional variant can contain one or more mutations (e.g., amino acid substitutions and/or deletions) in one or more of Domains 1-4 and Domain 6 of a wild-type BG2 protein (e.g., SEQ ID NO:3). Such a BG2 functional variant may comprise an amino acid sequence at least 80% identical (e.g., 85%, 90%, 95%, 98%, or higher) to the amino acid sequence of the wild-type counterpart, e.g., SEQ ID NO:3 or SEQ ID NO:4. The BG2 functional variant may have the whole region of Domain 1, Domain 2, Domain 3, Domain 4, or Domain 6 deleted.

### II. Transgenic Plant Overly Expressing a BG2 Gene

The “transgenic plant” described herein refers to a plant that comprises a transgene (such as a nucleic acid comprising an exogenous BG2 gene or an exogenous promoter) allowing for over-expressing of a BG2 gene in the transgenic plant.

The term transgene as used herein refers to a nucleic acid sequence which is introduced into a plant cell by experimental manipulations. In some embodiments, one or more cells of the transgenic plant carry the transgene. In other embodiments, the genome of the transgenic plant has been altered by the introduction of a transgene.

The transgenic plant described herein over-expresses a BG2 gene, which is also described herein, as compared to its wild-type counterpart. The term “over-express” or “over-expression” means that the transgenic plant exhibit an elevated level of a BG2 protein as compared to its wild-type counterpart, for example, the level of the BG2 in the transgenic plant is at least 20% (e.g., at least 30%, 50%, 70%, 90%, 2-fold, 5-fold, 10-fold, 20-fold, 50-fold, or 100-fold) higher relative to the level of the BG2 protein in the wild-type counterpart. In some instances, a transgenic plant described herein expresses an exogenous BG2 protein, which is not present in the wild-type counterpart.

The plants, as described herein, refer to a plurality of plant cells which are largely differentiated into a structure that is present at any stage of a plant's development. The plant described herein may be a full plant or a part thereof, including a fruit, shoot, stem, root, leaf, seed, panicle, flower petal, or similar structure. The plants may contain a plant tissue, which includes differentiated and undifferentiated tissues of plants including, but not limited to, roots, shoots, leaves, pollen, seeds, tumor tissue and various types of cells in culture (e.g., single cells, protoplasts, embryos, callus, protocorm-like bodies, and other types of cells). Plant tissue may be in planta, in organ culture, tissue culture, or cell culture. Similarly, plant cells may be cells in culture or may be part of a plant.

In some embodiments, the plant is an angiosperm. Angiosperms are flowering plants, sometimes referred to as angiospermae or magnoliophyta, that produce seeds and can be distinguished from the gymnosperms by characteristics including, but not limited to flowers, endosperm within the seeds, and the production of fruits that contain the seeds. Angiosperms are divided into dicotyledonous and monocotyledonous plants.

In certain embodiments, the plants as described herein are monocotyledonous plants. Monocotyledonous plants are flowering plants having embryos with one cotyledon or seed leaf, parallel leaf veins, and flower parts in multiples of three. Examples of monocots include, but are not limited to, maize, rice, maize, turfgrass, oat, wheat, barley, sorghum, orchid, iris, lily, onion, and palm. In some embodiments, the monocotyledonous plants are Zea mays, Sorghum bicolor, Setaria italica, Hordeum vulgare, Brachypodium distachyon, or Oryza sativa.

In other embodiments, the plants described herein are dicotyledonous plants. Dicotyledonous plants are flowering plants having embryos with two cotyledons or seed leafs, reticulated leaf veins and flower parts in multiples of fours or fives. Examples of dicots include, but are not limited to cotton, flax, grapevines, fruit, such as pomaceous fruit, but also stone fruit, vegetables, such as cucumbers, tomatoes, cabbage, and useful plants, such as soya beans, peanuts, potatoes, and also ornamental plants, such as annual and/or perennial trees and shrubs. In some embodiments, the dicotyledonous plants are Cucumis sativus, Ricinus communis, Solanum lycopersicum, Solanum tuberosum, Vitis vinifera, Populus trichocarpa, Arabidopsis thaliana, or Arabidopsis lyrata.

In certain embodiments, the plants can be crops. A crop is a cultivated plant that is harvested for food, clothing, livestock fodder, biofuel, medicine or any other use. Some non-limiting examples of crops include rice, maize, cotton, soybean, wheat, banana, potato, tomato, orchid, balloon flower, yams, sweet potato, cassava, rose, petunia, chrysanthemum, and lily.

In some embodiments, the transgene introduced into a plant as described herein comprises an exogenous promoter and is inserted upstream to the endogenous BG2 gene of a host plant such that the exogenous promoter is capable of driving the expression of the endogenous BG2 gene. The exogenous promoter may be a promoter from a different organism, e.g., a different plant. Alternatively, the exogenous promoter may be a promoter from the same host plant but not in operable linkage to the endogenous BG2 gene in nature.

In other embodiments, the transgene comprises an exogenous BG2 gene operably linked to a suitable promoter. Such a transgene is introduced into the plant to generate a transgenic plant that expresses the exogenous BG2 gene, e.g., a BG2 gene as described herein. It should be appreciated that a BG2 gene from one species may be overexpressed in a different species to achieve the same advantageous features as described herein.

As used herein, the term promoter, refers to a DNA sequence, which, when ligated to a nucleotide sequence of interest (e.g., upstream of an endogenous plant BG2 gene), is capable of controlling the transcription of the nucleotide sequence of interest (i.e., in operable linkage to the gene of interest) into mRNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription.

Promoters may be tissue specific or cell specific. A tissue specific promoter refers to a promoter that is capable of directing selective expression of a nucleotide sequence of interest to a specific type of tissue (e.g., leaves) in the relative absence of expression of the same nucleotide sequence of interest in a different type of tissue (e.g., roots). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. A cell type specific promoter refers to a promoter which is capable of directing selective expression of a nucleotide sequence of interest in a specific type of cell in the relative absence of expression of the same nucleotide sequence of interest in a different type of cell within the same tissue. A cell type specific promoter may also refer to a promoter capable of promoting selective expression of a nucleotide sequence of interest in a region within a single tissue. Cell type specificity of a promoter may be assessed using methods well known in the art, e.g., immuno-histochemical staining.

Promoters may be constitutive or regulatable. A constitutive promoter refers to a promoter that is capable of directing transcription of an operably linked nucleic acid sequence in the absence of a stimulus (e.g., heat shock, chemicals, light, or similar stimuli). Typically, constitutive promoters are capable of directing expression of a transgene in substantially any cell and any tissue. In contrast, a regulatable promoter is one which is capable of directing a level of transcription of an operably linked nucleic acid sequence in the presence of a stimulus (e.g., heat shock, chemicals, light, or similar stimuli) which is different from the level of transcription of the operably linked nucleic acid sequence in the absence of the stimulus. In some embodiments, the promoter operably linked to a BG2 gene is a constitutive promoter. In other embodiments, the promoter operably linked to a BG2 gene a regulatable promoter. Some non-limiting examples of regulatable promoters include, but are not limited to tissue specific promoters, growth stage-specific promoters, environment-inducible promoters, stress-inducible promoters, nutrient-inducible promoters, and chemical-inducible promoters. In certain embodiments, the promoter inserted upstream of the endogenous BG2 gene is a maize ubiquitin gene (Ubi) promoter, a rice actin promoter, a CaMV35S promoter, a rice glutelin promoter, a sweet potato sporamin promoter, a tomato E8 promoter, a rbcS1 promoter, or an α-amylase promoter.

Promoter sequences suitable for expression in plants are described in the art, e.g., WO 91/198696. These include non-constitutive promoters or constitutive promoters, such as, a nopaline synthetase and octopine synthetase promoters, a maize ubiquitin gene (Ubi) promoter, a rice actin promoter, a rice glutelin promoter, a sweet potato sporamin promoter, a tomato E8 promoter, a rbcS1 promoter, an α-amylase promoter, the cauliflower mosaic virus (CaMV) 19S and 35S promoters and the figwort mosaic virus (FMV) 35 promoter (see U.S. Pat. Nos. 5,352,605 and 6,051,753, both of which are hereby incorporated by reference). Promoters used may also be tissue specific promoters targeted for example to the endosperm, aleurone layer, embryo, pericarp, stem, leaves, tubers, roots, and the like.

Additional exemplary promoters that are suitable for use in constructing the transgenic plants as described herein include, but are not limited to, aleurone layer specific promoters. For example, the promoter may be a barley lipid transfer protein (Ltp2) promoter, a maize lipid transfer protein (AL9) promoter or a water rice protein (B3) promoter. In some embodiments, the promoter is an endosperm specific promoter. For example, the promoter may be a rice gliadin 4 a gene promoter, a rice 26 kDa globin promoter or a MEG1 endosperm-specific promoter e.g., WO 2005042745 Al. In certain embodiments, the promoter is an embryo specific promoter. For example the promoter may be an Esr promoter e.g., U.S. Pat. No. 7,071,378 B1. In certain embodiments, the promoter is a seed specific promoter. For example the promoter may be a pea β-phaseolin promoter (see e.g., Bustos et, 1989, Plant Cell 1: 839˜853), a rice glutelin gene (Glu-B1) promoter (see e.g., Washida etc., 1999, Plant Mol. Biol. 40: 1˜12) or a pea trypsin/chymotrypsin inhibitor gene (TI) promoter (see e.g., Welham and Domoney, 2000, Plant Sci. 159: 289˜299). In certain embodiments, the promoter is a pericarp specific promoter. For example, the promoter may be a maize cystatin promoter e.g., U.S. Pat. No. 8,481,811 B2. In certain embodiments the promoter is a root specific promoter. For example, the promoter may be a sweet potato MADS-box promoter e.g., CN 1842597 A. In yet other embodiments, the promoter is a stem-regulated, plant defense-inducing promoter. For example, the promoter may be an OMT promoter e.g., WO 2004062365 A2.

Enhancers may be included to increase and/or maximize transcription of the BG2 protein. These include, but are not limited to peptide export signal sequence, codon usage, introns, polyadenylation, and transcription termination sites (see WO 01/29242).

In some embodiments the vectors, described herein, have one or more selectable markers, such as prokaryote selectable markers. Such markers include resistance toward antibiotics such as ampicillin, tetracycline, kanamycin, and spectinomycin. Specific examples include but are not limited to streptomycin phosphotransferase (spt) gene coding for streptomycin resistance, neomycin phosphotransferase (nptII) gene encoding kanamycin or geneticin resistance, hygromycin phosphotransferase (hpt) gene encoding resistance to hygromycin.

In some embodiments, the transgenic plants, described herein, over-express BG2. As used herein, “expression” of a nucleic acid sequence refers to one or more of the following events: (1) production of an RNA transcript from a DNA sequence (e.g., by transcription); (2) processing of an RNA transcript (e.g., by splicing, editing, 5′cap formation, and/or 3′ end processing); (3) translation of an RNA transcript into a polypeptide or protein; and (4) post-translational modification of a polypeptide or protein. In some embodiments, production of a BG2 RNA transcript, in a BG2 transgenic plant, is at least 1.2 fold, at least 1.5 fold, at least 1.8 fold, at least 2 fold, at least 3 fold, at least 4 fold, at least 5 fold, at least 10 fold, at least 15 fold, at least 20 fold, at least 25 fold, at least 30 fold, at least 40 fold, at least 50 fold, at least 60 fold, at least 80 fold or at least 100 fold greater than the production of a BG2 RNA transcript of the wild-type counterpart. In other embodiments, production of a BG2 polypeptide, in a BG2 transgenic plant, is at least 1.2 fold, at least 1.5 fold, at least 1.8 fold, at least 2 fold, at least 3 fold, at least 4 fold, at least 5 fold, at least 10 fold, at least 15 fold, at least 20 fold, at least 25 fold, at least 30 fold, at least 40 fold, at least 50 fold, at least 60 fold, at least 80 fold or at least 100 fold greater than the production of a BG2 polypeptide of the wild-type counterpart.

Any of the transgenic plants described herein would exhibit improved growth rate as compared to its wild-type counterpart. Plant growth rate may refer to the mass of the plant, the height of the plant, the width of the plant, the shoot length, or the root length of the plant. In some embodiments, the mass, height, width, shoot length or root length of a transgenic plant that over-expresses a BG2 gene is at least 1%, at least 2%, at least 3%, at least 4%, at least 5%, at least 8%, at least 10%, at least 15%, at least 20%, at least 25%, at least 30%, at least 40%, at least 50%, at least 60%, at least 80%, or at least 100% greater than its wild-type counterpart. In other embodiments, the improved growth occurs within 10 days, within 20 days, within 30 days, within 60 days, within 90 days, within 120 days, or within 150 days after sowing.

Any of the transgenic plants described herein would also exhibit improved productivity, e.g., increasing an agriculturally desirable trait. For example, the agriculturally desirable trait may be increased grain yield, increased fruit yield, increased shoot weight, increased root weight, increased biomass, decreased time for flowering, decreased time for fruit formation, increased percentage of seed germination, increased quality of seed germination, decreased disease incidence, or increased disease resistance. In other embodiments the agriculturally desirable trait is increased grain size (e.g., length, width, thickness, or mass), increased panicle length, increased number of primary branches per panicle, increased number of secondary branches per panicle, or increased number of spikelet per panicle. In some embodiments, an agriculturally desirable trait of a transgenic plant that over-expresses a BG2 gene is at least 1%, at least 2%, at least 3%, at least 4%, at least 5%, at least 8%, at least 10%, at least 15%, at least 20%, at least 25%, at least 30%, at least 40%, at least 50%, at least 60%, at least 80%, or at least 100% greater than its wild-type counterpart. In other embodiments, the increased productivity occurs within 10 days, within 20 days, within 30 days, within 60 days, within 90 days, within 120 days, or within 150 days after sowing.

Further, the transgenic plant described herein may have improved stress tolerance (e.g., biotic stress or abiotic stress such as osmotic stress, salt, dehydration or heat). Biotic Stress can be stress that occurs as a result of damage done to plants by other living organisms, such as bacteria, viruses, fungi, parasites, beneficial and harmful insects, weeds, and cultivated or native plants. Abiotic stress can be negative impact of non-living factors on the living organisms in a specific environment. The non-living variable may influence the environment beyond its normal range of variation to adversely affect the population performance or individual physiology of the organism in a significant way.

In some embodiments, improving the stress tolerance of a plant refers to increasing the ability of a plant to survive under stress, which may be expressed as % survival as compared to the wild-type counterpart of the transgenic plant. In other embodiments, stress tolerance refers to the ability of a plant to survive under abiotic stress. Abiotic stress, as defined herein, refers to the negative impact of non-living factors on the plants in a specific environment. In some embodiments, the inventive methods improve the ability of plants to survive osmotic stress, salt stress, or dry air stress. Osmotic stress may be mimicked by exposure to polyethylene glycol. (PEG). In some embodiments osmotic stress is mimicked using 30% PEG₆₀₀₀. Salt stress may be mimicked by exposure to NaCl. In some embodiments, salt stress is mimicked using 250 mM NaCl. In some embodiments, the inventive methods improve the survival rate of a transgenic plant that over-expresses a BG2 gene by at least 1%, at least 2%, at least 3%, at least 4%, at least 5%, at least 8%, at least 10%, at least 15%, at least 20%, at least 25%, at least 30%, at least 40%, at least 50%, at least 60%, at least 80%, or at least 100% greater than its wild-type counterpart. In other embodiments, the increased survival rate is determined after 2 hours, after 4 hours, after 6 hours, after 8 hours, after 12 hours, after 24 hours, after 36 hours, after 2 days, after 4 days, after 8 days after 15 days, after 30 days, after 60 days, or after 90 days of exposure to stress. Such a abiotic-stress tolerant transgenic plant may be grown in an environment comprising an abiotic stress factor, such as those described herein.

Methods of Making Transgenic Plants that Over-Express a BG2 Gene

Any suitable methods known in the art can be used for preparing the transgenic plant described herein. For example, any of the transgenes described herein may be introduced into plants using a vector. A vector, as described herein, refers to any genetic element, such as a plasmid, phage, transposon, cosmid, chromosome, retrovirus, virion, or similar genetic element, which is capable of replication when associated with the proper control elements and which can transfer gene sequences into cells and/or between cells. Thus, this term includes cloning and expression vehicles, as well as viral vectors. In some embodiments, the vectors comprise sequences allowing for insertion into the genome of a plant (e.g., homologous sequences allowing for homologous recombination). In other embodiments, the vectors (e.g., comprising a BG2 gene that is operably linked to a promoter) may exist in a transgenic plant extrachromosomally (i.e., they are not integrated into the plant genome).

An expression vector, as referred to herein, is a recombinant DNA molecule containing a desired coding sequence (or coding sequences), such as the BG2 sequences, described above, and appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. It is not intended that the present invention be limited to particular expression vectors or expression vectors with particular elements.

A nucleic acid encoding a BG2 gene may be inserted, according to certain embodiments of the present disclosure, into an appropriate expression vector, i.e., a vector which contains the necessary elements for the transcription and translation of the inserted coding sequence, or in the case of an RNA viral vector, the necessary elements for replication and translation, as well as selectable markers. These include but are not limited to a promoter region, a signal sequence, 5′ untranslated sequences, initiation codon (depending upon whether or not the structural gene comes equipped with one), and transcription and translation termination sequences. Methods for obtaining such vectors are known in the art (see WO 01/29242 for review).

The development of plant virus gene vectors for expression of foreign genes (e.g., BG2 genes) in plants provides a means to provide high levels of gene expression within a short time.

Suitable viral replicons include double-stranded DNA from a virus having a double stranded DNA genome or replication intermediate. The excised viral DNA is capable of acting as a replicon or replication intermediate, either independently, or with factors supplied in trans. The viral DNA may or may not encode infectious viral particles and furthermore may contain insertions, deletions, substitutions, rearrangements or other modifications. The viral DNA may contain heterologous DNA, which is any non-viral DNA or DNA from a different virus. For example, the heterologous DNA may comprise an expression cassette for a protein or RNA of interest (e.g., a BG2 protein or BG2 RNA).

Super binary vectors carrying the vir genes of Agrobacterium strains A281 and A348 are useful for high efficiency transformation of monocots. However, even without the use of high efficiency vectors, it has been demonstrated that T-DNA is transferred to maize at an efficiency that results in systemic infection by viruses introduced by agroinfection, although tumors are not formed (Grimsley et al., (1989) Mol. Gen. Genet. 217:309-316). This is because integration of the T-DNA containing the viral genome is not required for viral multiplication, since the excised viral genome acts as an independent replicon.

Another Agrobacteria-mediated transient expression assay is based on Agrobacterium-mediated transformation of tobacco leaves in planta (Yang et al., (2000) The Plant J 22(6): 543-551). The method utilizes infiltration of agrobacteria carrying plasmid constructs into tobacco leaves, and is referred to as agroinfiltration; it has been utilized used to analyze in vivo expression of promoters and transcription factors in as little as 2-3 days. It also allows examination of effects of external stimuli such as pathogen infections and environmental stresses on promoter activity in situ.

The vectors constructed may be introduced into the plant host system using procedures known in the art (e.g., WO 01/29242 and WO 01/31045). The vectors may be modified to intermediate plant transformation plasmids that contain a region of homology to an Agrobacterium tumefaciens vector, a T-DNA border region from A. tumefaciens. In some embodiments, a T-DNA is the transferred DNA of the tumor-inducint (Ti) plasmid of some species of bacteria such as Agrobacterium tumefaciens and Agrobacterium rhizogenes. An Agrobacterium can transfer into a plant cell nucleus any DNA located on the T-DNA. This T-DNA is part of the wild-type Ti- (in case of Agrobacterium tumefaciens) or Ri-plasmid (in case of A. rhizogenes). Wild-type T-DNA carries the genes causing, after integration in the plant genome, crown gall tumors or the hairy root syndrome in case of infection with A. tumefaciens or A. rhizogenes, respectively. Also located on the wild-type Ti- or Ri-plasmids are vir genes (virulence genes) which are activated by plant phenolic compounds. Products of the vir genes are responsible for the transfer of the T-DNA into the eukaryotic genome. For transformation purposes, the T-DNA is disarmed (i.e. all disease-causing genes are removed) and vir genes are supplied either in trans on a helper plasmid (the T-DNA encompassing heterologous gene(s) is then located on a second binary plant transformation vector) or in cis in case of a co-integrate plant transformation vector. The heterologous genes of interest are cloned in between the two T-DNA 22 bp (in case of octopine Ti plasmids) or 25 bp (in case of nopaline Ti plasmids) imperfect border core sequences constituting to the right border (RB) and the left border (LB), that are the only in cis elements necessary to direct T-DNA processing. The border core sequences in RB and LB are organized as imperfect repeats. In some embodiments, the T-DNA, comprising a promoter, is inserted upstream of an endogenous plant BG2 gene.

Alternatively, the vectors used in the methods described herein may be Agrobacterium vectors. Methods for introducing the vectors include but are not limited to microinjection, velocity ballistic penetration by small particles with the nucleic acid either within the matrix of small beads or particles, or on the surface and electroporation. The vector may be introduced into a plant cell, tissue or organ. In a specific embodiment, once the presence of a transgene as described herein is ascertained, a plant may be regenerated using procedures known in the art. The presence of desired proteins may be screened using methods known in the art, preferably using screening assays where the biologically active site is detected in such a way as to produce a detectable signal. This signal may be produced directly or indirectly. Examples of such assays include ELISA or a radioimmunoassay.

Nucleic acids can also be introduced into plants by direct injection. Transient gene expression can be obtained by injection of the DNA into reproductive organs of a plant (see, for example, Pena et al., (1987) Nature, 325:274), such as by direct DNA transfer into pollen (see, for example, Zhou et al., (1983) Methods in Enzymology, 101:433; D. Hess (1987) Intern Rev. Cytol., 107:367; Luo et al., (1988) Plant Mol. Biol. Reporter, 6:165. DNA can also be injected directly into the cells of immature embryos (see, for example, Neuhaus et al., (1987) Theor. Appl. Genet: 75:30; and Benbrook et al., (1986) in Proceedings Bio Expo 1986, Butterworth, Stoneham, Mass., pp. 27 54).

An Agrobacterium, as referred to herein, is a soil-borne, Gram-negative, rod-shaped phytopathogenic bacterium which causes crown gall. The term “Agrobacterium” includes, but is not limited to, the strains Agrobacterium tumefaciens, (which typically causes crown gall in infected plants), and Agrobacterium rhizogens (which causes hairy root disease in infected host plants). Infection of a plant cell with Agrobacterium generally results in the production of opines (e.g., nopaline, agropine, octopine) by the infected cell. Thus, Agrobacterium strains which cause production of nopaline (e.g., strain LBA4301, C58, A208) are referred to as “nopaline-type” Agrobacteria; Agrobacterium strains which cause production of octopine (e.g., strain LBA4404, Ach5, B6) are referred to as “octopine-type” Agrobacteria; and Agrobacterium strains which cause production of agropine (e.g., strain EHA105, EHA101, A281) are referred to as “agropine-type” Agrobacteria.

Agrobacterium-mediated transformation is applicable to both dicots and monocots. Optimized methods and vectors for Agrobacterium-mediated transformation of plants in the family Graminae, such as rice and maize have been described (see, for example, Heath et al., (1997) Mol. Plant-Microbe Interact. 10:221-227; Hiei et al., (1994) Plant J. 6:271-282 and Ishida et al., (1996) Nat. Biotech. 14:745-750). The efficiency of maize transformation is affected by a variety of factors including the types and stages of tissue infected, the concentration of Agrobacterium, the tissue culture media, the Ti vectors and the maize genotype.

Another useful basic transformation protocol involves a combination of wounding by particle bombardment, followed by use of Agrobacterium for DNA delivery (see, for example, Bidney et al., (1992) Plant Mol. Biol. 18:301-313). Both intact meristem transformation and a split meristem transformation methods are also known (U.S. Pat. No. 6,300,545, hereby incorporated by reference).

Additional methods utilizing Agrobacteria include agroinfection and agroinfiltration. By inserting a viral genome into the T-DNA, Agrobacterium can be used to mediate the viral infection of plants (see, for example, U.S. Pat. No. 6,300,545, hereby incorporated by reference). Following transfer of the T-DNA to the plant cell, excision of the viral genome from the T-DNA (mobilization) is required for successful viral infection. This Agrobacterium-mediated method for introducing a virus into a plant host is known as agroinfection (see, for example, Grimsley, “Agroinfection” pp. 325-342, in Methods in Molecular Biology, vol 44: Agrobacterium Protocols, ed. Gartland and Davey, Humana Press, Inc., Totowa, N.J.; and Grimsley (1990) Physiol. Plant. 79:147-153).

The methods described herein include both stable and transient over-expression of the BG2 genes as described herein. Techniques for transforming a wide variety of higher plant species for transient expression of an expression cassette are well known [see, for example, Weising et al., Ann. Rev. Genet. 22:421-477(1988)]. Variables of different systems include type nucleic acid transferred (DNA, RNA, plasmid, viral), type of tissue transformed, means of introducing transgene(s), and conditions of transformation. For example, a nucleic acid construct may be introduced directly into a plant cell using techniques ranging from electroporation, PEG poration, particle bombardment, silicon fiber delivery, microinjection of plant cell protoplasts or embryogenic callus or other plant tissue, or Agrobacterium-mediated transformation [Hiei et al., Plant J. 6:271-282 (1994)]. Because transformation efficiencies are variable, internal standards (eg, 35S-Luc) are often used to standardize transformation efficiencies.

Expression constructs for transient assays include plasmids and viral vectors. A variety of plant viruses that can be employed as vectors are known in the art and include cauliflower mosaic virus (CaMV), geminivirus, brome mosaic virus, and tobacco mosaic virus.

Plant tissues suitable for transient expression include cultured cells, either intact or as protoplasts (in which the cell wall is removed), cultured tissue, cultured plants, and plant tissue such as leaves.

Some transient expression methods utilize gene transfer into plant cell protoplasts mediated by electroporation or polyethylene glycol (PEG). These methods require the preparation and culture of plant protoplasts, and involve creating pores in the protoplast through which nucleic acid is transferred into the interior of the protoplast.

Exemplary electroporation techniques are described in Fromm et al., Proc. Natl. Acad. Sci. 82: 5824 (1985). The introduction of DNA constructs using polyethylene glycol precipitation is described in Paszkowski et al., EMBO J. 3: 2717-2722 (1984). PEG-mediated transformation of tobacco protoplasts, which includes the steps of isolation, purification, and transformation of the protoplasts, are described in Lyck et al., (1997) Planta 202: 117-125 and Scharf et al., (1998) Mol Cell Biol 18: 2240-2251, and Kirschner et al., (2000) The Plant J 24(3): 397-411. These methods have been used, for example, to identify cis-acting elements in promoters activated by external stimuli, Abel and Theologis (1994) Plant J 5: 421-427; Hattori et al., (1992) Genes Dev 6: 609-618; Sablowski et al., (1994) EMBO J 13: 128-137; and Solano et al., (1995) EMBO J 14: 1773-1784), as well as for other gene expression studies (U.S. Pat. No. 6,376,747, hereby incorporated by reference).

Ballistic transformation techniques are described in Klein et al., (1987) Nature 327: 70-73. Biolistic transient transformation is used with suspension cells or plant organs. For example, it has been developed for use in Nicotiana tabacum leaves, Godon et al (1993) Biochimie 75(7): 591-595. It has also been used in investigating plant promoters, (Baum et al., (1997) Plant J 12: 463-469; Stromvik et al., (1999) Plant Mol Biol 41(2): 217-31, Tuerck and Fromm (1994) Plant Cell 6: 1655-1663; and U.S. Pat. No. 5,847,102, hereby incorporated by reference), and to characterize transcription factors (Goff et al., (1990) EMBO J 9: 2517-2522; Gubler et al., (1999) Plant J 17: 1-9; and Sainz et al., (1997) Plant Cell 9: 611-625).

Other methods allow visualization of transient expression of genes in situ, such as with onion epidermal peels, in which GFP expression in various cellular compartments was observed (Scott et al., (1999) Biotechniques 26(6): 1128-1132.

Transgenic plants that stably over-express a BG2 gene as described herein may be selected by examining genome insertion of a transgene described herein following conventional methology.

Without further elaboration, it is believed that the above description has adequately enabled the present invention. The following examples are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

### BG2 Improves Plant Growth, Stress Tolerance, and Productivity

### Materials and Methods

### (i) Plant Materials

Rice (Oryza sativa ssp. japonica cv. Tainung 67) was used in this study. Plants were grown under greenhouse conditions at 26° C. to 30° C. Plants were cultured in half-strength Murashige and Skoog (MS) medium containing 0.3% phytagel. For yield analysis in field, plants were grown in the experimental farm of National Chung Hsing University in Taichung.

### (ii) Phylogenetic Analysis of BG2-Related Protein

Protein sequences showing similarity to BG2 protein were retrieved using the public BLAST server on the National Center for Biotechnology Information website (http://www.ncbi.nlm.nih.gov/). The phylogenetic analysis was performed using BioEdit CLUSTAL W analysis tool, using the neighbor-joining method, and the resulting phylogenetic tree was visualized using the MEGA 6.0 software.

(iii) RNA Extraction

For mRNA expression profile, total RNA was extracted from various tissues during rice development with the TRIZol kit (Invitrogen, USA). Plant tissue was grinded with liquid nitrogen and mixed with 1 mL TRIZol reagent. After incubation for at least 5 minutes at room temperature, 200 μL chloroform was added and mixed well. The mixture was centrifuged 13,000×g for 15 minutes at 4° C. Aqueous phase was transferred to another new tube, then added 0.5 mL isopropanol. After mixed well by gently inverting, the mixture was incubated at −20° C. for at least 30 minutes. After incubation, the tubes were centrifuged 13,000 rpm for 30 minutes at 4° C. The supernatants were removed and RNA pellets were washed with 75% EtOH. RNA pellets were dissolved in DEPC-H2O. After full dissolved, 1 μL DNaseI (Invitrogen, USA) and 1 μL RNasin (Invitrogen, USA) was added and incubated at 37° C. for 1 hour. After incubation, 450 μL DEPC-H2O and 500 μL phenol (pH 4.2) (Invitrogen, USA) was added and mixed well by gently inverting. The tubes were centrifuged 13,000×g for 10 minutes at 4° C. The supernatants were transferred to new tubes and 200 μL chloroform was added then mixed well. The mixture was centrifuged 13,000 rpm for 15 minutes at 4° C. After repeated twice, the tubes were centrifuged 13,000 rpm for 30 minutes at 4° C. The supernatants were removed and RNA pellets were washed with 75% EtOH. RNA pellets were dissolved in DEPC-H2O.

### (iv) Reverse Transcription Polymerase Chain Reaction (RT-PCR)

For cDNA synthesis, 13 μL mixture containing 5 μg RNA, 1 μL oligo-dT, 1 μL 10 mM dNTP, and DEPC-H2O was heated at 65° C. for 5 minutes by PCR machine and incubated on ice for at least 1 minute. Mixture containing 4 μL 5× First-Strand Buffer (Invitrogen, USA), 1 μL 0.1 M DTT, 1 μL RNase inhibitor and 1 μL SuperScript™ III Reverse Transcriptase was added. After incubation at 50° C. for 60 minutes and 70° C. for 15 minutes, the cDNA was stored at −20° C. and 10× diluted for further use.

For PCR analysis, 25 μL of mixture containing 1 μL diluted cDNA, μL 10×PCR buffer, μL 10 mM dNTP, 0.5 μL 10 μM gene-specific forward and reverse primers (Appendix 4), 0.5 μL VioTaq polymerase (Viogene, Taiwan) was subjected to PCR reaction. PCR reaction was performed with initial denaturation at 95° C. for 5 minutes followed by 25-35 cycles of incubation at 95° C. for 30 seconds, 55-60° C. for 30 seconds, 72° C. for 30-40 seconds, and a final extension at 72° C. for 5 minutes.

### (v) Histochemical GUS Staining Assay

Samples (pBG2::GUS) were collected from various stages freshly and then stained in a solution of 1 mM 5-bromo-4-chloro-3-indolyl-β-D-glucuronic acid (X-gluc), 50 mM NaPO4 buffer, 0.1 mM each K3Fe(CN)6/K4Fe(CN)6, 0.05% Triton X-100, 1 mM EDTA, 20% methanol, and incubated at 37° C. in the dark for 16 h. After GUS staining, chlorophyll was removed using 70% ethanol at 65° C.

### (iv) Scanning Electronic Microscope Analysis

Grains of wild-type rice, two independent Ubi: BG2 transgenic lines and D16/BG2act were attached to a stub. The samples were frozen with liquid nitrogen slush then transferred to a sample preparation chamber at −160° C. After 5 minutes, when the temperature rose to −130° C., the samples were fractured and etched for 10 minutes at −85. After coating at −130° C., the samples were transferred to the SEM chamber and observed at −160 with a cryo scanning electron microscope (FEI Quanta 200 SEM/Quorum Cryo System PP2000TR FEI).

### Results

### (i) BG2 Encodes an Unknown Function Protein and is Conserved Among Angiosperms

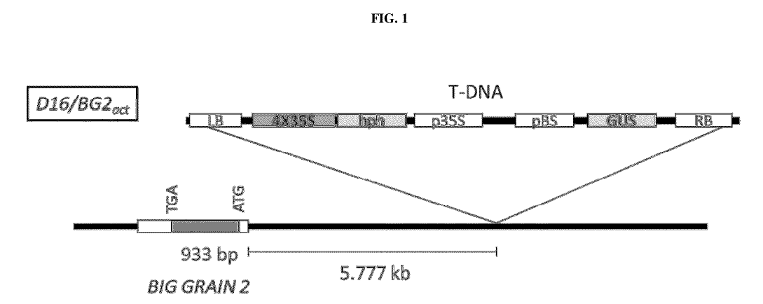

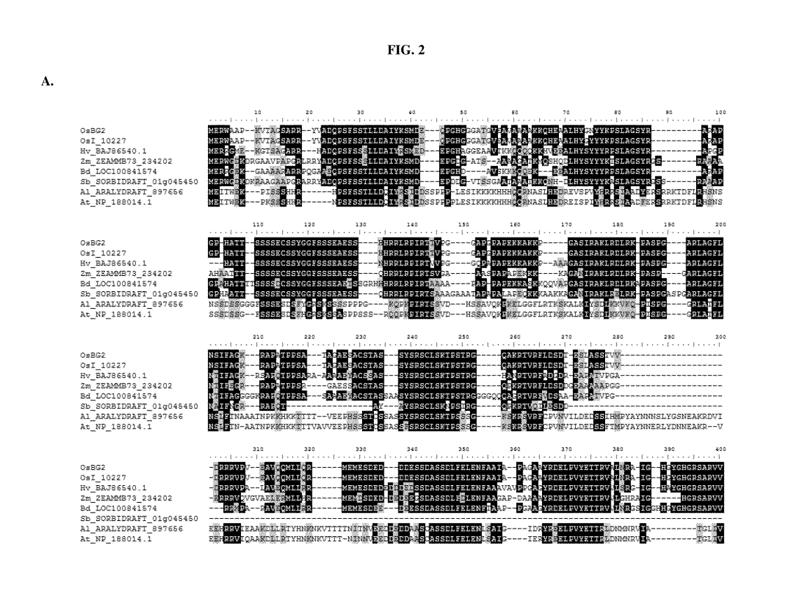

The BG2 gene (Os03g0175800) has no intron, and the full-length cDNA reported by the Rice Genome Annotation Project database (http://rice.plantbiology.msu.edu/) from Oryza sativa ssp. japonica cv. Nipponbare has a length of 1527 bp, with a 129 bp 5′-UTR, 933 bp coding sequence and 465 bp 3′-UTR (FIG. 1). BG2 encodes 311 amino acids, which is identified as a putative expressed protein (MW=32803.4; pI=9.7064). Analysis of the predicted sequence of BG2 protein by online program InterPro (http://www.ebi.ac.uk/interpro/) revealed no putative functional domains, and found that it contains no putative sequence motifs that define a specific subcellular localization or extracellular secretion. To obtain more information of BG2 protein function, we further analyzed the amino acid sequence of BG2 with another online program, the Eukaryotic Linear Motif resource for Functional Sites in Proteins (ELM) (http://elm.eu.org/). Interestingly, although no function-known domains were predicted in BG2 amino acid sequence, several functional motifs such as protein phosphorylation recognition sites have been found. Examination of BG2 protein sequence revealed 15 putative GSK3 kinase phosphorylation recognition sites, S/TXXXS/T (where S/T is Ser or Thr and X is any amino acid), which has the highest score of probability (0.0268).

To analyze the phylogenetic relationship and find putative functional regions of BG2, a BLAST search using the BG2 amino acid sequence was performed with the online BLAST service (National Center for Biotechnology Information, NCBI, http://blast.ncbi.nlm.nih.gov/Blast.cgi). Six conserved regions of BG2 amino acid sequence were found (FIG. 2). The rice genome encodes one BG2-related protein (LOC_Os10g25810) that has shared 50% sequence identity with BG2. We further named it as BG2-LIKE (BG2L). Orthologs of BG2 and BG2L exist in other cereals, including barley, maize, and sorghum. Moderately similar proteins are conserved among several dicotyledonous plants, such as Arabidopsis, Populus, and Vitis. Functions of most BG2-related proteins have not yet been investigated, except four putative proteins predicted in three organisms: putative protein TPRXL (tetra-peptide repeat homeobox-like)-like (identity=69%) in Setaria italic, corepressor interacting with RBJ 1-like (identity=29%) and protein AF-9-like (identity=26%) in Cicer arietinum, and micronuclear linker histone polyprotein-like (identity=32%) in Glycine max. However, BG2 and its orthologous proteins comprise a family that is conserved between monocots and dicots. Phylogenetic analysis revealed that BG2-like proteins can be approximately classified into two groups: monocot-specific and dicot-specific groups (FIG. 3). These results suggested that BG2 encodes an expressed protein with unknown function which is conserved in Angiosperms. To identify putative function of BG2 protein domains, the full length amino acid of BG2 was separated into six regions and searched databases for homologous sequences of these six putative domains (FIG. 2). Region 1 and 6 only displayed in angiosperm, indicating it may be plant specific. Region 2 to 5 shared sequence identity to proteins with diverse functions, indicating that BG2 may function specifically in Angiosperms with several novel regulating domains.

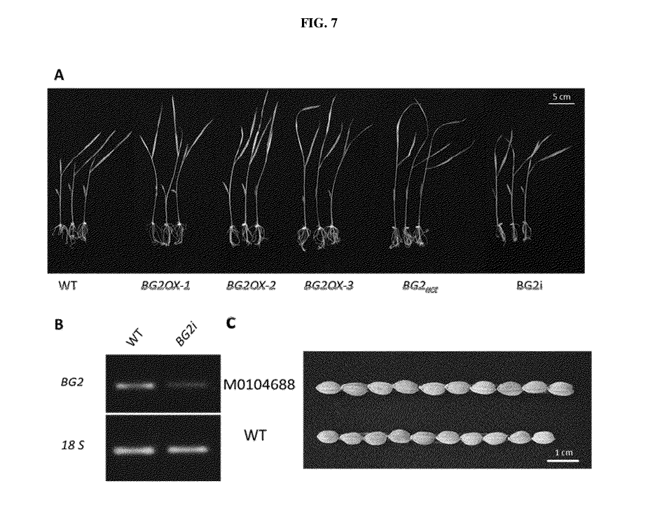

10 independent BG2 RNAi lines were screened and 3 of those lines were significantly down-regulated. These knockdown BG2 plants showed only minor phenotypic differences compared to the wild-type and activation of BG2L (M0104688) also displayed larger grain (FIG. 7), indicating it may be due to the functional redundancy of BG2 and BG2L.

### (ii) Characterization of the BG2 Overexpression Lines

T0 transgenic plants that overexpress BG2 driven by maize ubiquitin promoter shows similar phenotype to D16/BG2act, indicating that it also leads to an increase in grain size. To further confirm the phenotype, transgenic line BG2OX-3 was chosen for T1, T2 and T3 plants propagation and observed phenotypes revealed during the whole growth stages. The BG2OX mutant exhibited visible phenotypes during vegetative stages, such as increase in plant height, root length and tiller angle. BG2OX plants were slightly higher than wild-type plants both in the greenhouse and the field.

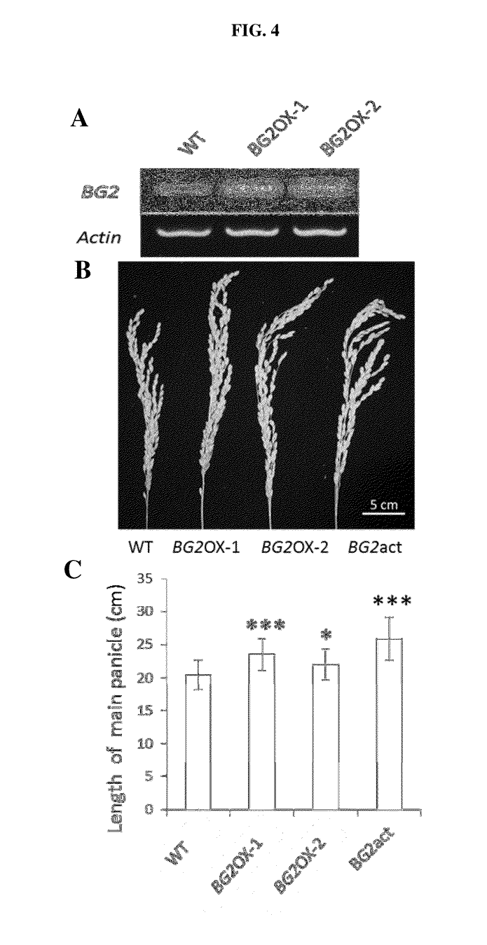

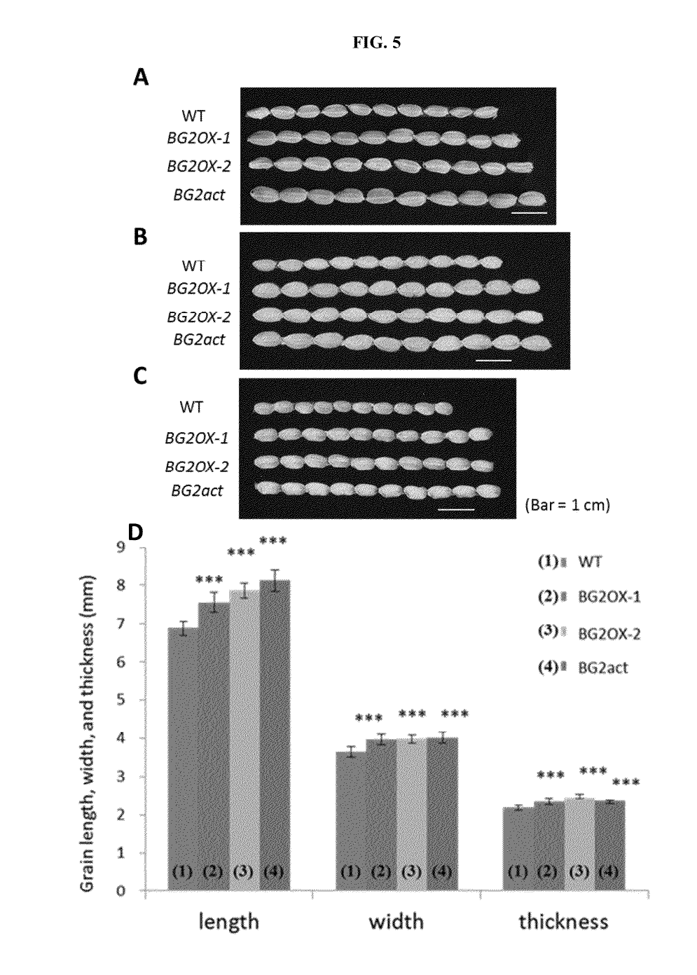

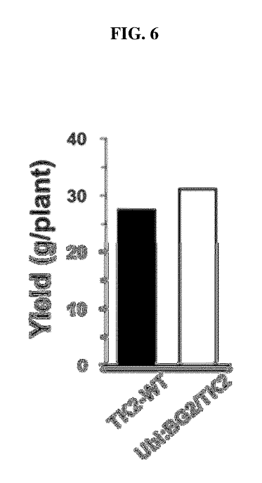

To further examine the alteration in panicle structure, grain size, panicle length, number of primary branches per panicle, number of secondary branches per panicle, and number of spikelet per panicle were measured of BG2OX and the wild-type plants. A slight decrease of the panicle number, the number of primary and secondary branches per panicle, and the number of spikelet per panicle was observed in BG2act and three BG2OX lines, while the plant height in field, the panicle length, the grain size (length, width, and thickness) and 100-grain weight increase (FIG. 4, 5; Table 1). However, the number of primary branches per panicle of BG2act was increased (Table 1). The 100-grain weights of the BG2OX line was significantly greater than the wild-type, and the grain yield per plant of BG2OX-3 is slightly enhanced compared to the wild-type (Table 1), indicating that overexpression of BG2 in wild-type plants still has potential to enhance grain yield. Overexpression of BG2 under the control of Ubi promoter also led to slightly higher grain yield (FIG. 6; Table 1)). Taken collectively, these results indicate that BG2 increases productivity by influencing panicle structure, panicle length, grain size and weight in rice.

TABLE 1
Comparison of yield traits of BG2act and three independent transgenic lines
overexpressing BG2 with wild-type.

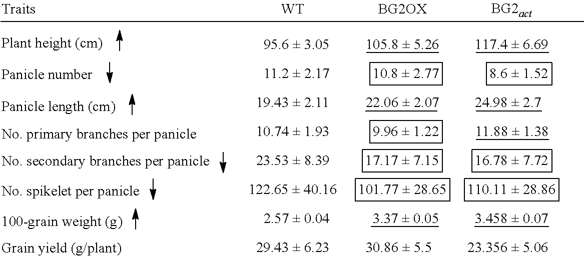

Plant height, panicle length, 100-grain weight are increased in BG2 overexpression and D16/BG2act lines. Underlined values indicate parameters increased compared to the wild-type. Boxed in values indicate parameters decreased compared to the wild-type. Values are shown with mean±standard deviation.

(iii) Histological Analysis of BG2OX Lines

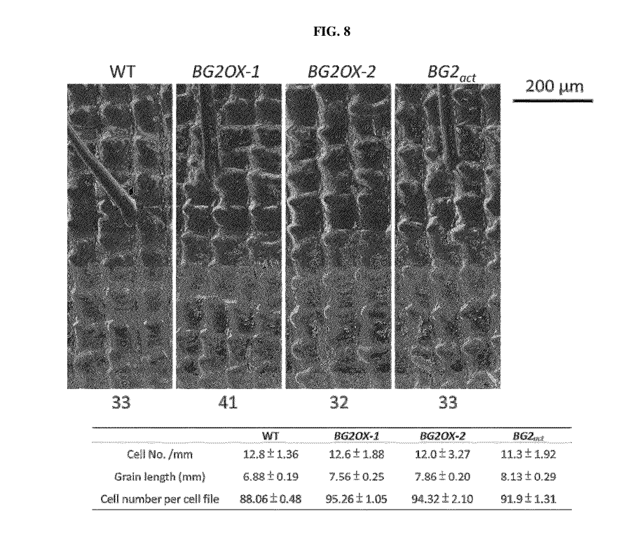

To elucidate that the overall organ increasing is caused by influence of cell proliferation or cell expansion, tissue section and scanning electron microscope (SEM) observation were carried out to examine the cellular alterations in BG2OX lines. The most dramatic phenotypic alteration in BG2OX lines was in grains, the cellular basis of the increase in grain size was focused on first. By measuring the size of mature wild-type and BG2OX grains, it was found that the average length of outer epidermal cells of BG2OX lemmas was indistinguishable from that of wild-type lemmas and total number of cells increased both in BG2OX and BG2act plant compared to the wild-type (FIG. 8). These results indicated that the increase in cell number was responsible for the increase of grain size in BG2OX plants.

### (iv) BG2 is Preferentially Expressed in the Developing Tissues.

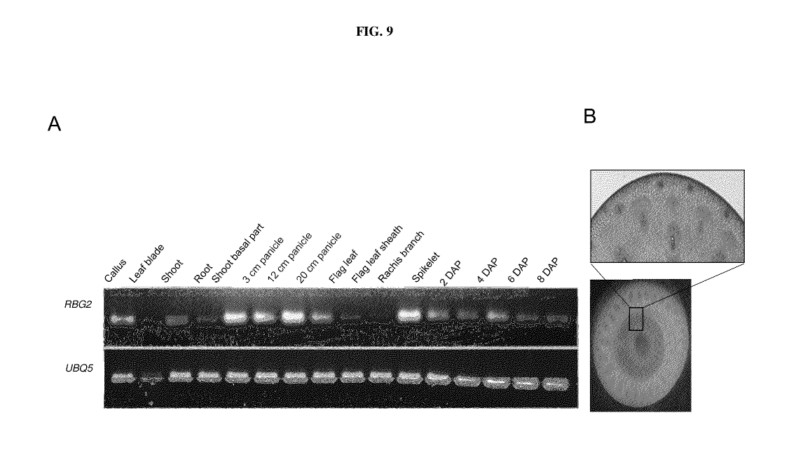

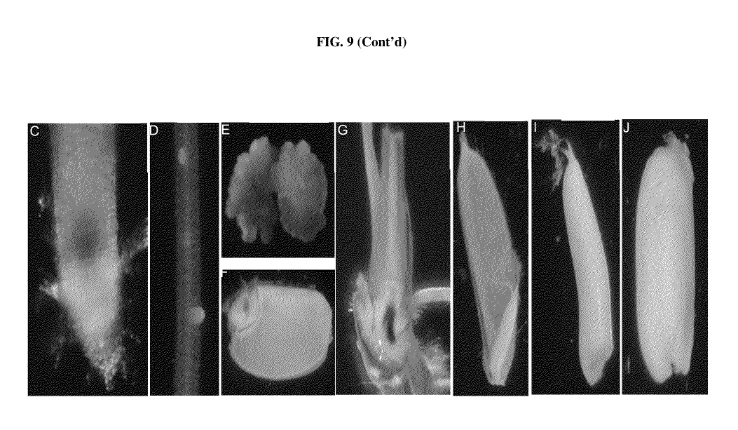

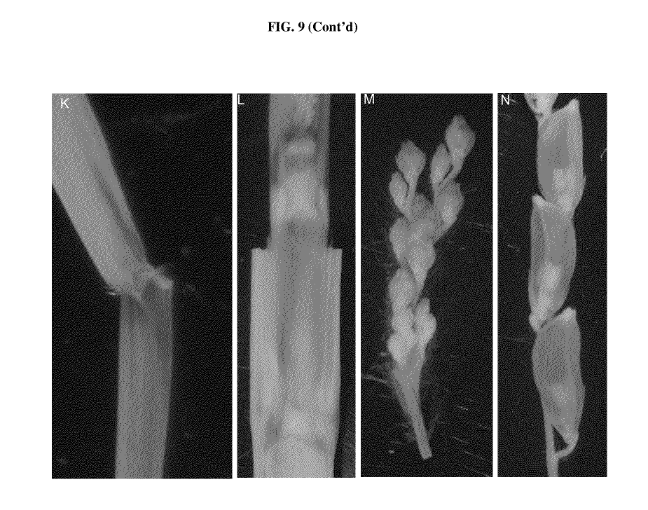

To investigate the expression profile of endogenous BG2, the GENEVESTIGATOR database (genevestigator.com/gv/) and Rice eFP Brower (bar.utoronto.ca/efprice/cgi-bin/efpWeb.cgi) were searched, which contains a large amount of microarray data covering the entire life cycle of rice plant. BG2 was expressed in various tissues, including calli, seed, leaf, and root, especially expressed higher in shoot apical meristem and inflorescence tissues. Expression of BG2 is comparatively lower in reproductive tissues such as pollen and stigma. Tissues covering all of the rice development stages were collected from wild-type rice to extract RNA for RT-PCR analysis. BG2 was preferentially expressed in the developing tissues such as shoot apex and developing panicle (FIG. 9). Moderate expression in calli, shoot, and panicle during grain filling stages was also observed (FIG. 9). Low or no signal was detected in mature tissues (FIG. 9), similar to the previous results predicted by online databases.

To further examine the spatial and temporal expression of BG2, a construct was produced in which the BG2 promoter (approximately 2.5 kb upstream of the translation start site) was fused to the β-glucuronidase gene (GUS) to transform wild-type plants. GUS activity was detected higher in the calli, leaf primordia and scutellum of geminating seeds, leaf intercalary meristem near laminar joint, internode during elongation, shoot apical meristem, vascular bundle and developing panicles, whereas tissues such as mature leaf have moderate expression (FIG. 9). Weak or no signals were observed in root, stamen, and developing seed (FIG. 9). GUS signals were detected in lateral root primordial but not in root apical meristem (FIG. 9). These observations were consistent with results from RT-PCR.

### (v) BG2 Confers Abiotic Stress Tolerance to Transgenic Rice.

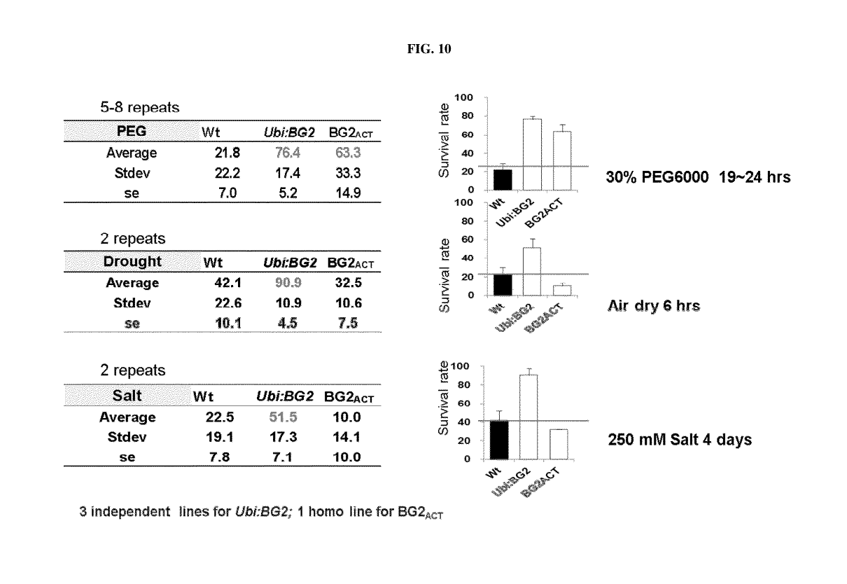

It was also found that BG2 could confer abiotic stress tolerance to transgenic rice. Treatment of transgenic rice expressing Ubi:BG2 with 30% PEG (which mimics osmotic stress), air-dry and 250 mM salt (NaCl) demonstrated that survival rates of transgenic plants were significantly greater than the wild type (FIG. 10). However, the survival rate of the T-DNA-tagged BG2 mutant (BG2_{act}) was greater only under the treatment of PEG, but not of air dry and salt, as compared with the wild type.

Further, twenty one-day-old seedlings of wild-type (WT) and BG2 transgenic (BG2_{Act} rice were treated with treated with PEG (30% PEG 6000 for 18 hours), NaCl (250 mM for four days), dehydration (for six hours), or heat (42° C. for four days). The plants were then recovered in 0.5× Kimura solution for 7 days after the treatment. The survival rates of the treated plants were determined. As shown in FIG. 14, BG2 enhanced tolerance to abiotic stresses, such as osmotic stress, salt, dehydration, and heat.

### (vi) Overexpression of BG2 Increases Organ Size and Enhances Tolerance of Abiotic Stress Factors in Arabidopsis.

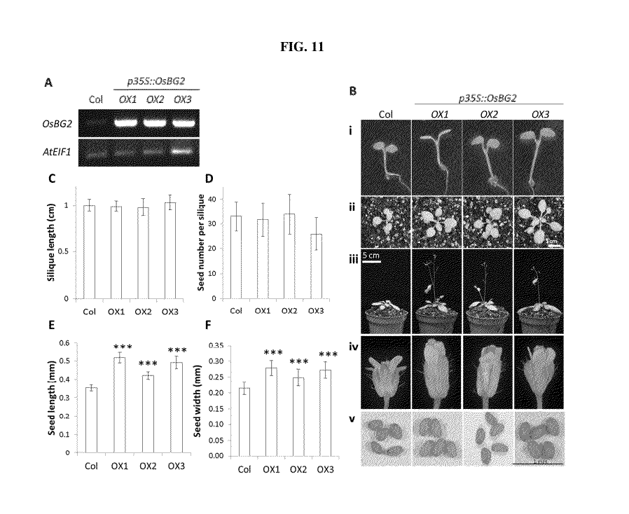

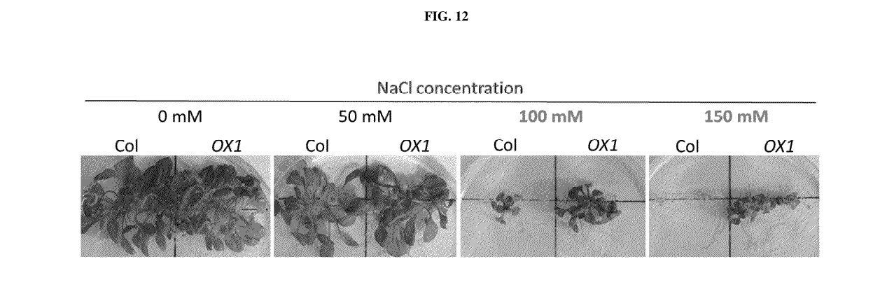

To investigate whether BG2 has similar functions in dicots as in rice, three independent transgenic plants were generated that express CaMV35S::BG2 in Arabidopsis thaliana. All three independent lines displayed phenotypes of increased organ size, including larger petals, leaves, and seeds, but no significant change in silique length and seed number per silique, as compared with the wild type (FIG. 11). Treatment of these transgenic plants with 50 mM, 100 mM, and 150 mM salt (NaCl) demonstrated that salt tolerance of transgenic plants were significantly greater than the wild type (FIG. 12).

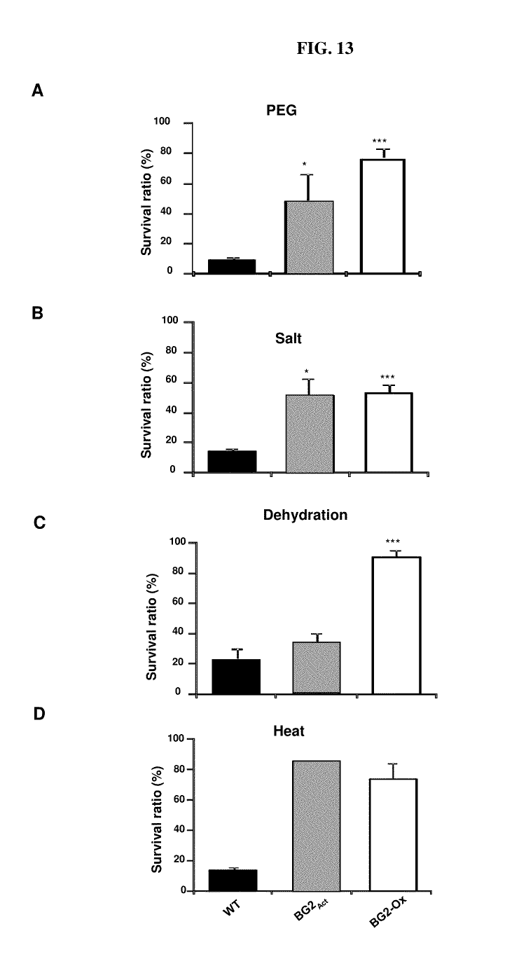

Further, twenty one-day-old seedlings of wild-type (WT) plants and plants that overly express BG2 (BG2-OX) with treated with PEG (30% PEG 6000 for 18 hours), NaCl (250 mM for four days), dehydration (for six hours), or heat (42° C. for four days). The plants were then recovered in 0.5× Kimura solution for 7 days and the survival rates thereof were determined. As shown in FIG. 13, overexpression of BG2 significantly enhanced tolerance to abiotic stresses, such as osmotic stress, salt, dehydration, and heat.

### Examples 2

### Deletion Mutants of BG2 and Functions Thereof

Sequence alignment of BG2 versus BG2-like proteins in seven plant species revealed that there are six conserved domains in this protein family. FIG. 2, panel A. Deletion mutants of BG2 were constructed to investigate the impact of the six conserved domains on the activity of BG2 for improving grain size. These six domains are indicated in SEQ ID NO:3 above (boldfaced and underlined). A schematic illustration of the constructs carrying truncated BG2 genes is provided in FIG. 2, panel B. These truncated BG2 constructs were introduced into rice for expression under the control of the Ubi promoter following the methods described in Example 1 above.

The sizes of grains from the wild-type or transgenic plants in TNG67 (T2 generation) and Kitaake (T1 generation) background were measured. The grain sizes of the transgenic plants were compared with those of the wild-type rice. The results were shown in FIG. 2, panel C. Domain 5 appears important in the activity of BG2 to increase grain size as deletion of this domain abrogated this activity of BG2. On the other hand, deletion of the other domains (Domains 1-4 and 6) showed little impact on BG2's activity in increasing grain size. Transgenic rice expressing BG2 with deletions of Domain 2 and Domain 6 in Kitaake background produced larger grains as compared to transgenic rice expressing the full-length BG2.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. An isolated nucleic acid encoding a Big Grain 2 (BG2) mutant, wherein the BG2 mutant contains one or more mutations within one or more of the regions corresponding to Domain 1, Domain 2, Domain 3, Domain 4, and/or Domain 6 in SEQ ID NO:3.

2. The isolated nucleic acid of claim 1, wherein the one or more mutations are deletion(s).

3. The isolated nucleic acid of claim 1, wherein the whole region corresponding to Domain 1, Domain 2, Domain 3, Domain 4, or Domain 6 is deleted in the BG2 mutant.

4. A transgenic plant, which expresses the BG2 mutant encoded by the nucleic acid of claim 1.

5. The transgenic plant of claim 4, wherein the plant is a monocotyledonous plant or a dicotyledonous plant.

6. The transgenic plant of claim 5, wherein the plant is a monocotyledonous plant selected from the group consisting of Zea mays, Sorghum bicolor, Setaria italica, Hordeum vulgare, Brachypodium distachyon, Oryza sativa, Triticum spp., and Saccharum spp.

7. The transgenic plan of claim 5, wherein the plant is a dicotyledonous plant selected from the group consisting of Cucumis sativus, Ricinus communis, Solanum lycopersicum, Solanum tuberosum, Vitis vinifera, Populus trichocarpa, Arabidopsis thaliana, Arabidopsis lyrata, and Platycodon grandiflorus.

8. The transgenic plant of claim 5, wherein the plant is a crop.

9. The transgenic plant of claim 8, wherein the crop is rice, maize, wheat, barley, sugarcane, banana, cotton, soybean, pea, potato, tomato, brassica, orchid, balloon flower, yam, sweet potato, cassava, rose, petunia, chrysanthemum, lily, or carnation.

10. The transgenic plant of claim 5, wherein the plant is an angiosperm.

11. The transgenic plant of claim 4, wherein the nucleic acid encoding the BG2 mutant is in operable linkage to a promoter, which is a tissue-specific promoter, a growth stage-specific promoter, an environment-inducible promoter, a stress-inducible promoter, a nutrient-inducible prompter, or a chemical-inducible promoter.

12. The transgenic plant of claim 4, wherein the nucleic acid encoding the BG2 mutant is in operable linkage to a promoter, which is a maize ubiquitin gene (Ubi) promoter, a rice actin promoter, a CaMV35S promoter, a rice glutelin promoter, a sweet potato sporamin promoter, a tomato E8 promoter, a rbcS1 promoter, or an α-amylase promoter.

13. A method of improving plant growth, architecture, organ size, productivity, or stress tolerance, the method comprising expressing a Big Grain 2 (BG2) mutant in a plant, wherein the BG2 mutant is encoded by the nucleic acid of claim 1.

14. A method for enhancing survival rate of a plant in an environment comprising an abiotic stress factor, the method comprising:
(i) providing a transgenic plant, which overly expresses a Big Grain 2 (BG2) gene as relative to its wild-type counterpart; and
(ii) growing the transgenic plant in an environment comprising an abiotic stress factor.

15. The method of claim 14, wherein the plant is a monocotyledonous plant or a dicotyledonous plant.

16. The method of claim 14, wherein the plant is a monocotyledonous plant selected from the group consisting of Zea mays, Sorghum bicolor, Setaria italica, Hordeum vulgare, Brachypodium distachyon, Oryza sativa, Triticum spp., and Saccharum spp.

17. The method of claim 14, wherein the plant is a dicotyledonous plant selected from the group consisting of Cucumis sativus, Ricinus communis, Solanum lycopersicum, Solanum tuberosum, Vitis vinifera, Populus trichocarpa, Arabidopsis thaliana, Arabidopsis lyrata, and Platycodon grandiflorus.

18. The method of claim 14, wherein the plant is a crop.

19. The method of claim 18, wherein the crop is rice, maize, wheat, barley, sugarcane, banana, cotton, soybean, pea, potato, tomato, brassica, orchid, balloon flower, yam, sweet potato, cassava, rose, petunia, chrysanthemum, lily, or carnation.

20. The method of claim 14, wherein the plant is an angiosperm.

21. The method of claim 14, wherein the transgenic plant comprises an exogenous promoter located upstream to the endogenous BG2 gene or an exogenous BG2 gene, which is in operable linkage to a promoter.

22. The method of claim 14, wherein the abiotic stress factor is osmotic stress, salt, dehydration, or heat.


SEQUENCE LISTING (11 sequences)

SEQ ID NO: 1 - DNA, 933 bp, Oryza sativa Japonica
atggagaggtgggcggcgcccaaggtgacggctggttcggcgaggcggtacgtcgccgaccagccgtctttctcatccacgctgctcgacgcgatatacaagtcgatggacgagcagccgggtcacggcggcggcgccaccggggtggaggcggtggctgcggcggccaagaagcagcacgaggcggccctgcactatgggaactactacaagccgtcgctcgcggggagctaccgggcgcgcgcgccgggtccgcacgccacgacgtcgagctcgtcggagtgttctagctacggcgggttctcgtcgtccgaggcggagtcgtcgcaccaccgacgcctccgccccatccgcacgactgtccccggtggtgcgccggggcccgcgccggagaagaaggccaagaagcccggggcctccatacgcgccaagctcagggacctccgcaagccggcttcccccggcgcgcgcctcgcgggcttcctcaactccatcttcgccggcaagcgcgcgccggccacgccgccctcggcgacggccggggcggagtccgcgtgctcgacggcgtcgtcctactcccgctcctgcctgagcaagacgccgtcgacgcgcgggcaggcgaagcggaccgtgcggttcttggacagcgacacggagtccctggcgtcgtcgacggtggtcgaccgccgcagggtgcccgtggaggcggtgcagcagatgctgctccagcggatggagatggagagcgacgaggacgacgacgagagcagcgacgcgagctccgacctgttcgagctcgagaacttcgccgccattgctcccgccggggccgcgtaccgggacgagctgccggtgtacgagacgaccagagtggcgctgaaccgcgccattggccatgggtatggccatggacggagcgccagagttgtctga

SEQ ID NO: 2 - DNA, 1014 bp, Oryza sativa Japonica
atgcgcgacatggagatgaggtgggcggcgccggcgccggcggcgagggggagggggagggcgaggcggcgggcgcctgaccagccgtcgttctcgtcgacgctgctcgacgccatctgcgactccatggacgagggcggcgaggacggccggacaagaaacgcggcgagtgcggcggccaagaagaggcaggaggcggcgaacagctaccactactactactgctacaagccgtcgctggccgccagctacagggcggcgccggcgctgggttccacggcggattgccccgggaggggctacttctcgtcgtccgaggtggagtactcgcttcgccggctccgccccatccgcacctccgccgccggtggcgcgggagacggcgcggcggtcgcgcggaagcagcggcatgagcagccggatgtggagaagacggcgaagacgaagccgggctccgcctcggcccgcgcgtgccgcaggccggcgtcgcccggcgcgcggctcgccagcttgctcaactccatcttctccggcaagcgtccatccgcgcagcgcccggcgtgttcgccggactacccggagcccgcgtgctcgacggcgccaccgtcgtcgtcgtcgtcgtacgcgcgccgcccctgccacgccaagacgccgcgcaccccccccaccaccaccaccacggcgagggcgcggccgagccggagcaggaccgtccggttcctggacatcgacggcaaggtcgcggtggccgcggccgtcgccggctgccgccgaattccggtcatggaggtggaggccgacaccgacgacggaggtgaggagagcagcgacgcgagctcggacctgttcgagctcgacagcctcgcggccattgctccggcgggtggtcgcgacggctcctacggggacgagctgccggtgtacgggaccaccggagttgggatccgccgcgacattggccgccgccgtccgtacggccatgctccttgtcggagctggagtagggctgtctag

SEQ ID NO: 3 - protein, 310 aa, Oryza sativa Japonica
MERWAAPKVTAGSARRYVADQPSFSSTLLDAIYKSMDEQPGHGGGATGVEAVAAAAKKQHEAALHYGNYYKPSLAGSYRARAPGPHATTSSSSECSSYGGFSSSEAESSHHRRLRPIRTTVPGGAPGPAPEKKAKKPGASIRAKLRDLRKPASPGARLAGFLNSIFAGKRAPATPPSATAGAESACSTASSYSRSCLSKTPSTRGQAKRTVRFLDSDTESLASSTVVDRRRVPVEAVQQMLLQRMEMESDEDDDESSDASSDLFELENFAAIAPAGAAYRDELPVYETTRVALNRAIGHGYGHGRSARVV

SEQ ID NO: 4 - protein, 337 aa, Oryza sativa Japonica
MRDMEMRWAAPAPAARGRGRARRRAPDQPSFSSTLLDAICDSMDEGGEDGRTRNAASAAAKKRQEAANSYHYYYCYKPSLAASYRAAPALGSTADCPGRGYFSSSEVEYSLRRLRPIRTSAAGGAGDGAAVARKQRHEQPDVEKTAKTKPGSASARACRRPASPGARLASLLNSIFSGKRPSAQRPACSPDYPEPACSTAPPSSSSSYARRPCHAKTPRTPPTTTTTARARPSRSRTVRFLDIDGKVAVAAAVAGCRRIPVMEVEADTDDGGEESSDASSDLFELDSLAAIAPAGGRDGSYGDELPVYGTTGVGIRRDIGRRRPYGHAPCRSWSRAV

SEQ ID NO: 5 - protein, 310 aa, Oryza sativa
MERWAAPKVTAGSARRYVADQPSFSSTLLDAIYKSMDEQPGHGGGATGVEAVAAAAKKQHEAALHYGYYYKPSLAGSYRARAPGPHATTSSSSECSSYGGFSSSEAESSHHRRLRPIRTTVPGGAPGPAPEKKAKKPGASIRAKLRDLRKPASPGARLAGFLNSIFAGKRAPATPPSATAGAESACSTASSYSRSCLSKTPSTRGQAKRTVRFLDSDTESLASSTVVDRRRVPVEAVQQMLLQRMEMESDEDDDESSDASSDLFELENFAAIAPAGAAYRDELPVYETTRVALNRAIGHGYGHGRSARVV

SEQ ID NO: 6 - protein, 318 aa, Hordeum vulgare
MERRGMEKGTSAGARRHADQPSFSSSLLDAIYRSMEDEPGHAGGEAAVTKKQQQQKKHVEEALHYSYYYRPSLAGSYRAPGPHATTSSSSECSSYGGFSSSEAESSNHRRLRPIRTVVPGGQPAPAPEKKAKKPAAAGASIRAKLRDLRKPASPGARLAGFLNTIFAGKRSAPQTPPSARAAAAAEYACSSASSYSRSCLSKTPSTRGHANRTVRFADGDREAPATVPGADRRRVPALAVEQMLLRRMEMESDEDEDDDEDSSDASSDLFELENFAAAVAVPPGAGYRDELPVYETTRVVLSRGIGHAYGHGRSARVV

SEQ ID NO: 7 - protein, 316 aa, Zea mays
MERWGDKDRGAAVPAPGRLRRYADQPSFSSSLLDAIYKSMDEPGDGATSAAAAGATKMQSHQDLHYSYYYKTSLAGSYRGSRAAAAAHAATTTSSSSECSSYGGFSSSEAESSQHRRLRPIRTSVGAAASPAPAPEKKKKAGANIRAKLRDLRKPASPGARLAGFLNTIFSGRRAPATPPSRGAESSACSTASSYSRSCLSKTPSTRGQPKRTVRFLDSDDGEAAAAAPGGERRRVQVGVAELERMLLHRMEMDSDEDDEDEEGSDASSDLFDLENFAAGAPDAAAAYRDELPVYETTRVVLGHRAIGHGRSARVV

SEQ ID NO: 8 - protein, 327 aa, Brachypodium distachyon
MERRGEKGAAAARARRPQGAAEQPSFSSTLLDAIYKSMDEPGHDAVSKKKQQEKEEALHYSYYYRPSLAGSYRARAPGPAHATTTTSSSSDCSSYGGFSSSEAETSSGRHHHRRLRPIRTAAAAPAPPAPEKKASKKQQVAPGASIRAKLRDLRKAPASPGARLAGFLNTIFAGGGGKRAPQTPPSASAAAEYACSTASSAASYSRSCLSKTPSTRGGGGQQQAGRTVRFVDSAAEAPATVPGRRMPARAVEQMLLRRMEMESDEEDEESSDASSDLFELENFTAAPPGAAGYRDELPVYETTRVVLNRGSIGGHHGYGHGRSARVV

SEQ ID NO: 9 - protein, 223 aa, Sorghum bicolor
MERWGDKDKRAAGAAPGRARRYADQPSFSSTLLDAIYKSMDEPDDGVTSSGAATAAATKKQNHDLHYSYYYKASLAGSYRGSSRAAAPGPHAATTSSSSECSSYGGFSSSEAESSQHRRLRPIRTSAAAGAAATAPAPALAPEQKKKAAKKAGANIRAKLRELRKPASPGASPGARLAGFLNAIFNGRRAPQTAYAYSRSCLSKNPSIRGQPKRTVQILESDD

SEQ ID NO: 10 - protein, 358 aa, Arabidopsis lyrata
MEITWEKPISSSHHRHPSFSSTLLDQIYRSIDDSSPPPLESIKKKKHHHQQRNASLHEDREVSPVYHRRSIAADYERSRRKTDFLRHSNSNSSDSSGGGFSSSESDSFYGRSKSSSSPPPGKQPKPIRTSSVDHSSAVQKPKELGGFLRTKSKALKIYSDLKKVKQPISPGGRLATFLNSLFTNAAATNPKKHKKTTTTVEEPHSSSTCSSASSYSRSCLSKTPSSSGKSKRSVRFCPVNVILDEDSSIHMPYAYNNNSLYGSNEAKRDVIEEHRRVIEAAKDLLRTYHNKNKVTTTTNITNVEEDDEDDAASCASSDLFELENLSAIGIDRYREELPVYETTRLDNMNRVIATGLIV

SEQ ID NO: 11 - protein, 357 aa, Arabidopsis thaliana
MEITWEKPKSSSHHRNPSFSSTLLDQIYRSIDDSSPPPPLESIKKKKHHHQQRNASLHEDREISPIYHRRSIAADFERSRRKTDFLRHSNSSSSDSSGFSSSESDSFHGRSKSSASPPSSSRQQPKPIRTSSVDHSSAVQKPKELGGFLRTKSKALKIYSDLKKVKQPISPGGRLATFLNSLFTNAATNPKKHKKTTTVAVVEEPHSSSTCSSASSFSRSCLSKTPSSSGKSKRSVRFCPVNVILDEDSSFTMPYAYNNERLYDNNEAKRVEEHRRVIQAAKDLLRTYHNKNKVTTTNINNVEEDDEDDAASCASSDLFELENLSAIGIERYREELPVYETTRLDNMNRVIATGLIV